SEQUENCE LISTING (71 sequences)

SEQ ID NO: 1 - skipped

SEQ ID NO: 2 - DNA, 836 bp, homo sapiens
ctcgtgcgaattcggcagaactctgtctgacctgacaagccacctcaagtggacaaggcacttaccaacagagattgctgatttgctccttaagcaagagattcactgccgctaagcatggctcagaccaactcgttcttcatgctgatctcctccctgatgttcctgtctctgagccaaggccaggagtcccagacagagctgcctaatccccgaatcagctgcccagaaggcaccaatgcctatcgctcctactgctactactttaatgaagaccctgagacctgggttgatgcagatctctattgccagaacatgaattcaggcaacctggtgtctgtgctcacccaggcggagggtgccttcgtggcctcactgattaaggagagtagcactgatgacagcaatgtctggattggcctccatgacccaaaaaagaaccgccgctggcactggagtagtgggtccctggtctcctacaagtcctgggacactggatccccgagcagtgctaatgctggctactgtgcaagcctgacttcatgctcaggattcaagaaatggaaggatgaatcttgtgagaagaagttctcctttgtttgcaagttcaaaaactagaggaagctgaaaaatggatgtctagaactggtcctgcaattactatgaagtcaaaaattaaactagactatgtctccaactcagttcagaccatctcctccctaatgagtttgcatcgctgatcttcagtaccttcacctgtctcagtctctagagccctgaaaaataaaaacaaacttatttttatccagtgaacaaagagagggaggagaagagac

SEQ ID NO: 3 - skipped

SEQ ID NO: 4 - DNA, 871 bp, homo sapiens
acctgctggcccctggacacctctgtcaccatgtggttcctggttctgtgcctcgccctgtccctgggggggactggtgctgcgcccccgattcagtcccggattgtgggaggctgggagtgtgagcagcattcccagccctggcaggcggctctgtaccatttcagcactttccagtgtgggggcatcctggtgcaccgccagtgggtgctcacagctgctcattgcatcagcgacaattaccagctctggctgggtcgccacaacttgtttgacgacgaaaacacagcccagtttgttcatgtcagtgagagcttcccacaccctggcttcaacatgagcctcctggagaaccacacccgccaagcagacgaggactacagccacgacctcatgctgctccgcctgacagagcctgctgataccatcacagacgctgtgaaggtcgtggagttgcccacccaggaacccgaagtggggagcacctgtttggcttccggctggggcagcatcgaaccagagaatttctcatttccagatgatctccagtgtgtggacctcaaaatcctgcctaatgatgagtgcgaaaaagcccacgtccagaaggtgacagacttcatgctgtgtgtcggacacctggaaggtggcaaagacacctgtgtgggtgattcagggggcccgctgatgtgtgatggtgtgctccaaggtgtcacatcatggggctacgtcccttgtggcacccccaataagccttctgtcgccgtcagagtgctgtcttatgtgaagtggatcgaggacaccatagcggagaactcctgaacgcccagccctgtcccctacccccagtaaaatcaaatgtgcatccaaaaaa

SEQ ID NO: 5 - skipped

SEQ ID NO: 6 - DNA, 644 bp, homo sapiens
ctgactataagactatacctgagactggtcatctcagttcttttctcaccttgactgcaagatgaaactccttgtgctagctgtgctgctcacagtggccgccgccgacagcggcatcagccctcgggccgtgtggcagttccgcaaaatgatcaagtgcgtgatcccggggagtgaccccttcttggaatacaacaactacggctgctactgtggcttggggggctcaggcacccccgtggatgaactggacaagtgctgccagacacatgacaactgctatgaccaggccaagaagctggacagctgtaaatttctgctggacaacccgtacacccacacctattcatactcgtgctctggctcggcaatcacctgtagcagcaaaaacaaagagtgtgaggccttcatttgcaactgcgaccgcaacgctgccatctgcttttcaaaagctccatataacaaggcacacaagaacctggacaccaagaagtattgtcagagttgaatatcacctctcaaaagcatcacctctatctgcctcatctcacactgtactctccaataaagcaccttgttgaaagacaaaagaaaaaaaaagaaaaaaagaaaaaagaaaggaaagggaagcagggaaaatggga

SEQ ID NO: 7 - DNA, 723 bp, homo sapiens
attttgcgaggctaatggtgcgtaaaagggctggtgagatctgggggcgcctcctagcctgacgtcagagagagggaggggtttaaaacagagggagacggttgagagcacacaagccgctttaggagcgaggttcggagccatcgctgctgcctgctgatccgcgcctagagtttgaccagccactctccagctcggctttcgcggcgccgagatgctgtcctgccgcctccagtgcgcgctggctgcgctgtccatcgtcctggccctgggctgtgtcaccggcgctccctcggaccccagactccgtcagtttctgcagaagtccctggctgctgccgcggggaagcaggaactggccaagtacttcttggcagagctgctgtctgaacccaaccagacggagaatgatgccctggaacctgaagatctgtcccaggctgctgagcaggatgaaatgaggcttgagctgcagagatctgctaactcaaacccggctatggcaccccgagaacgcaaagctggctgcaagaatttcttctggaagactttcacatcctgttagctttcttaactagtattgtccatatcagacctctgatccctcgcccccacaccccatctctcttccctaatcctccaagtcttcagcgagacccttgcattagaaactgaaaactgtaaatacaaaataaaattatggtgaaattatgagaaagaaaa

SEQ ID NO: 8 - skipped

SEQ ID NO: 9 - DNA, 801 bp, homo sapiens
gtcagagctgagaaaggtgtgaggggtatataagagctggattactagttagcaaatgagggggtaaatattccagtggatacaagcttggactcttttcttgaagctttctttctatcagaagcatttgctgatattgctgacattgaaacattaaaagaaaatttgagaagcaatgggcatcctgaagctgcaagtatttctcattgtgctctctgttgcattgaaccatctgaaagctacacccattgaaagtcatcaggtggaaaagcggaaatgcaacactgccacatgtgcaacgcagcgcctggcaaattttttagttcattccagcaacaactttggtgccattctctcatctaccaacgtgggatccaatacatatggcaagaggaatgcagtagaggttttaaagagagagccactgaattacttgcccctttagaggacaatgtaactctatagttattgttttatgttctagtgatttcctgtataatttaacagtgcccttttcatctccagtgtgaatatatggtctgtgtgtctgatgtttgttgctaggacatataccttctcaaaagattgttttatatgtagtactaactaaggtcccataataaaaagatagtatcttttaaaatgaaatgtttttgctatagatttgtattttaaaacataagaacgtcattttgggacctatatctcagtggcacaggtttaagaacgaaggagaaaaaggtagttggacccttggaaggtgtgcgcggctgatgtgttgtccgcggggcggaatccggc

SEQ ID NO: 10 - skipped

SEQ ID NO: 11 - DNA, 608 bp, homo sapiens
gaaagaaactggttgtttcattcctggctggagagagtagaaaagagaatgaagatggagtaacatacatttgtgacattcccagaacctggaggccaggctatgacacagagtcaatcaataaccagggagatctgtgatatagcccagtaggtggggccttgctgccatctgccatatgacccttccagtcccaggcttctgaagagacgtggtaagtgcggtgcagttttcaactgacctctggacgcagaacttcagccatgaaggtaacaggcatctttcttctcagtgccttggccctgttgagtctatctggtaacactggagctgactccctgggaagagaggccaaatgttacaatgaacttaatggatgcaccaagatatatgaccctgtctgtgggactgatggaaatacttatcccaatgaatgcgtgttatgttttgaaaatcggaaacgccagacttctatcctcattcaaaaatctgggccttgctgagaaccaaggttttgaaatcccatcaggtcaccgcgaggcctgactggccttattgttgaataaatgtatctgaatatccaaaaaaaaaaagcgaaaaaagaggaa

SEQ ID NO: 12 - DNA, 892 bp, homo sapiens
gtcttctcctctttttttgctttttttcttggaattttctgagagatctttattctttattcaggatgtgggatcgatcagcactgccagctgggccttggttctagtggcttgctatggtctcctcaatccagtcgatgaaggcggagactcgagtgaacaccgtgggcttcctgcgggtgttgcagccaaaggcagaaacaaagctggtcacgccatggacctgccagccaccatcctctgtggggcagttgaggggtcctccagagtcaccgttgcagccggagcggatgtcccctccagcacacaccatggtcttcttcacggaggaaccccaccagttccacctggagcagtgttcatagtccaccacgggcagcagggcctcctgcagcttgtctgggagtggcccgttggtatagagacggccccagccggtgatgtagcagggtgtctcgttgggaaggatgtcaccagcgggagggagtgaggcgagctggacggcgtctcccagctgggcgctgcgtgagagcttgatgagggcgatgtcattgccacaggccacacacgagcggttccagagtggatgcacaaagaggtccccagagttgatggggatcacctgctcggggccctccttcacagcacggtcgtactcgcccaacaccacctggtaggtccaggagctcgagatgcagtggccggcagtcacaacccagtcgggggcgatgaggctaccgccacacgtgtggtagaagcttccacttttctcatactgcagggaaacctgccagggccagctgtaggggaccgcatcctcaccattgacaacgcggctggaagggcgagaggaaggtgggccatagcctgaggcaacggccacaaggagcgg

SEQ ID NO: 13 - skipped

SEQ ID NO: 14 - DNA, 229 bp, homo sapiens
gcagtaatcaacgcccgcggtccaccactcctgcccaccgccccccagccactggcacgaagcatctctggctgggcaacactgcgaccggcgccgactacccagacagcgcaggcctgatgctccggcgaccagcaagtgtgaacctcctacccggaaagattaccacaacatgttccgtgtgggctccttaggggcaaggacacatgtcagtgattctggtgccctg

SEQ ID NO: 15 - DNA, 885 bp, homo sapiens
cgcaagcgcatggcttccctctggctcctctcctgcttctcccttgtgggggccgcctttggctgcggggtccccgccatccaccctgtgctcagcggcctgtccaggatcgtgaatggggaggacgccgtccccggctcctggccctggcaggtgtccctgcaggacaaaaccggcttccacttctgcgggggctccctcatcagcgaggactgggtggtcaccgctgcccactgcggggtcaggacctccgacgtggtcgtggctggggagtttgaccagggctctgacgaggagaacatccaggtcctgaagatcgccaaggtcttcaagaaccccaagttcagcattctgaccgtgaacaatgacatcaccctgctgaagctggccacacctgcccgcttctcccagacagtgtccgccgtgtgcctgcccagcgccgacgacgacttccccgcggggacactgtgtgccaccacaggctggggcaagaccaagtacaacgccaacaagacccctgacaagctgcagcaggcagccctgcccctcctgtccaatgccgaatgcaagaagtcctggggcaggaggatcaccgacgtgatgatctgtgccggggccagtggcgtctcctcctgcatgggcgactctggcggccccctggtctgccaaaaggatggagcctggaccctggtgggcattgtgtcctggggcagcgacacctgctccacctccagccctggcgtgtacgcccgtgtcaccaagctcataccttgggtgcagaagatcctggctgccaactgagcccgcggctccctccgaccctgctccccacagagcctcagtaaacccatggaacacaaaaaaaagggaggaaaaaaaaacata

SEQ ID NO: 16 - DNA, 656 bp, homo sapiens
ggatctcgtgaggtcagggccccttttatagccccatcaccacagctggctctgtctcccgccacccacaccagctgtcccactcaccatggagaagatcctgatcctcctgcttgtcgccctctctgtggcctatgcagctcctggcccccgggggatcattatcaacctggagaacggtgagctctgcatgaatagtgcccagtgtaagagcaattgctgccagcattcaagtgcgctgggcctggcccgctgcacatccatggccagcgagaacagcgagtgctctgtcaagacgctctatgggatttactacaagtgtccctgtgagcgtggcctgacctgtgagggagacaagaccatcgtgggctccatcaccaacaccaactttggcatctgccatgacgctggacgctccaagcagtgagactgcccacccactcccacacctagcccagaatgctgtaggccactaggcgcaggggcatctctcccctgctccagcgcatctcccgggctggccacctccttgaccagcatatctgttttctgattgcgctcttcacaattaaaggcctcctgcaaacctttaaaaaaaaaaagagaagaagaaaaaaaaaaggaaaaagaagaaaggaggagaagagaaggaggaa

SEQ ID NO: 17 - DNA, 105 bp, homo sapiens
tgtctgcgctgctccagcgcatctgccgggctggccacctccttgagcagcatatctgggttctgattgcgctcttcacaattaaaggcgtcctgcagaccttaa

SEQ ID NO: 18 - DNA, 1746 bp, homo sapiens
ctgtgaccagccccgtccaggctagtgcctgcaggaacattctggaggtaagtggctttcacaccactgctgccagaaggtatgtgttgggctgatgggacctatcatagtagctctgagatcttggacaagccacttagcttccctgagcccccagggttgtgtgagagtgcacaagttggtttacataaagagcttagtataagtctgacatgtgagtttcataaacgtaagcgacaattacgataatttcaattagggatggtgggggagggatgcacagagaagcaagcttgttggttcctgactcttcccaagggtcaataatttatcgttctagttttttgcctggtttcctcattcgccttcccataaagtgtcagtaacactgaaggaagaggtcccttgacctccatgagctcagaaggtagggtggctgcatggttaagcaaattgactctggaaatcagatctggatttgagatacacctacatacacatacacataaacatctgtatggttttgtttttttgccaaagagaaatcaaacccatgccatctacaaaaacaccctctccttggtcaatgatttcatcatcagagacaccatcctcaacatcaacttccaatgtgcctacccactggacatgaaagtcagcctccaagctgccttgcagcccattgtaagttccctgaacgtcagtgtggacgggaatggagagttcattgtcaggatggccctcttccaagaccagaactacacgaatccttacgaaggggatgcagttgaactgtctgttgagtccgtgctgtatgtgggtgccatcttggaacaaggggacacctcccggtttaacctggtgttgaggaactgctatgccacccccactgaagacaaggctgaccttgtgaagtatttcatcatcagaaacagctgctcaaatcagcgtgattccaccatccacgtggaggagaatgggcagtcctcggaaagccggttctcagttcagatgttcatgtttgctggacattatgacctagttttcctgcattgtgagattcatctctgtgattctcttaatgaacagtgccagccttcttgctcaagaagtcaagtccgcagtgaagtaccggccatcgacctagcccgggttctagatttggggcccatcactcggagaggtgcacagtctcccggtgtcatgaatggaacccctagcactgcagggttcctggtggcctggcctatggtcctcctgactgtcctcctggcttggctgttctgagagctccgctgagcatctggccttgaagtttgtgttcttccctctggcaatggctcccttcagcacttctgctttccactccaattcacacaggcttggtattaacagaatcaaggccaggctaggttaggaaaagggaagagctttcaccttctttaaaactctcggctgggcgcagtggctcatgcctgtaatcccagcattttgggaggctgaggcaggtggatcacctgaggtcagcagttcaaaatcagcctggccaaaatgctgaaactccgtctctactaaaaatacaaaaattagccaggcatggtggcaggcgcctgtaatcccagctactcgggaggccaaggcaggagaattgctcgaactcagggggtggaggttgcagtgagttgagattgtgccattgcactccagcctgggcaacagagcaagactctgtctcaggaaaaaaaa

SEQ ID NO: 19 - DNA, 785 bp, homo sapiens
ggaggagaaaaaggtagtttgaccttggtaaattgtaaacagttaataatggagttattctgacatgagaaattcagtaattgggaccaggcgcggtggttcttgcctgtaatccccagcactttgggagcccgaggcaggcagatcacaagttcaggagttcgagaccagcctgaccaacatggtgaaaccctgtctttactaaaaatacaaaaattagccgggggtggtgacatgtgcctgtaaatcccagtaactcaggaggtaaggcaggagaatcgcttaaacccaggaggcggaggttgcagtgagccgagattgcaccactgcactccagcctgggtggcagagtgagactcgtctcaaaaaaaagaaagaaaattagtaattgtaagtacccctgataagcaaattagtaattgtcaatacccctgttaagcaattcctttttgcagtatatttctgaaatgacagaatgctgttttaaaaacaaagaaataaaatcctgctcctgactcggtcaaaatattttttaaagtctattgtttgttgtgcttgctggtactaagaggcaatttaaaagtataaaactgctttgtatccatgagggtttcattgtgtgttagcagcagtgagcttctattaaatgtatatgtcatttattttgtttaagtggctttcagcaaacctcagtcatattcttatgcagggtattgcgaaacaacttgtgttctattaatcgtgtcttcaattaaaagaccacagacttctggaaaaaaaaaaaa

SEQ ID NO: 20 - skipped

SEQ ID NO: 21 - DNA, 901 bp, homo sapiens
ccacgatgttgctgctcagcctgaccctaagcctggttctcctcggctcctcctggggctgcggcattcctgccatcaaaccggcactgagcttcagccagaggattgtcaacggggagaatgcagtgttgggctcctggccctggcaggtgtccctgcaggacagcagcggcttccacttctgcggtggttctctcatcagccagtcctgggtggtcactgctgcccactgcaatgtcagccctggccgccattttgttgtcctgggcgagtatgaccgatcatcaaacgcagagcccttgcaggttctgtccgtctctcgggccattacacaccctagctggaactctaccaccatgaacaatgacgtgacgctgctgaagctcgcctcgccagcccagtacacaacacgcatctcgccagtttgcctggcatcctcaaacgaggctctgactgaaggcctcacgtgtgtcaccaccggctggggtcgcctcagtggcgtgggcaatgtgacaccagcacgtctgcagcaggtggctttgcccctggtcactgtgaatcagtgccggcagtactggggctcaagtatcactgactccatgatctgtgcaggtggcgcaggtgcctcctcgtgccagggtgactccggaggccctcttgtctgccagaagggaaacacatgggtgcttattggtattgtctcctggggcaccaaaaactgcaatgtgcgcgcacctgctgtgtatactcgagttagcaagttcagcacctggatcaaccaggtcatagcctacaactgagctcaccacaggccctccccagctcaacccattaaagacccaggccctgtcccatcatgaaaaaaaaaaagggaagaaaaaaaaagggcggccgcaaactg

SEQ ID NO: 22-23 - skipped (2 entries)

SEQ ID NO: 24 - DNA, 560 bp, homo sapiens
gggacggctcagcgacgccacggccagcagcgctcgcgtcctccccagcaacagttactcaaagctaatcagatagcgaaagaagcaggagagcaagtcaagaaatacggtgaaggagtccttcccaaagttgtctaggtccttccgcgccggtgcctggtcttcgtcgtcaacaccatggacagctcccgggaaccgactctggggcgcttggacgccgctggcttctggcaggtctggcagcgctttgatgcggatgaaaaaggttacatagaagagaaggaactcgatgctttctttctccacatgttgatgaaactgggtactgatgacacggtcatgaaagcaaatttgcacaaggtgaaacagcagtttatgactacccaagatgcctctaaagatggtcgcattcggatgaaagagcttgctggtatgttcttatctgaggatgaaaactttcttctgctctttcgccgggaaaacccactggacaagcagcgtggagtttatgcagatttggcgcaaatatgacgctgacagcagtggctttatatcagctg

SEQ ID NO: 25 - DNA, 565 bp, homo sapiens
gtccccgctgcgcccgctgctgctggccctggcccttgcctccgtgccttgcgcccagggcgcctgccccgcctccgccgacctcaagcactcggacgggacgcgcacttgcgccaagctctatgacaagagcgacccctactatgagaactgctgcgggggcgccgagctgtcgctggagtcgggcgcagacctgccctacctgccctccaactgggccaacaccgcctcctcacttgtggtggccccgcgctgcgagctcaccgtgtggtcccggcaaggcaaggcgggcaagacgcacaagttctctgccggcacctacccgcgcctggaggagtaccgccggggcatcttaggagactggtccaacgctatctccgcgctctactgcaggtgcagctgatgcattgctggtctctcatctgcagcttccacagagtgccaagcccctcactcagcccatccctgggctctgctccggggccccaagacccaggaggaggagcgttctgcctgccccctcccacctcccctgcaatacagcctttgtgcagttgtccacgtc

SEQ ID NO: 26 - skipped

SEQ ID NO: 27 - DNA, 553 bp, homo sapiens
cttgtaagaaggctcatgccattgaccctcttaattctctcctgtttggcggagtgacaatggcggaggctgaaggcttgatccagatggaagctgtgaaagtgaaaacattaaagtctttgacggaacctccagcaatgggcctctgctagggcaagtctgcagtaaaaacgactatgttcctgtatttgaatcatcatccagtacattgacgtttcaaatagttactgactcagcaagaattcaaagaactgtctttgtcttctactacttcttctctcctaacatctctattccaaactgtggcggttacctggataccttggaaggatccttcaccagccccaattacccaaagccgcatcctgagctggcttattgtgtgtggcacatacaagtggagaaagattacaagataaaactaaacttcaaagagattttcctagaaatagacaaacagtgcaaatttgattttcttgccatctatgatggccccttcaacaactttgggctgattgggcaagtctgtgggcgtgtggattgccaacttgga

SEQ ID NO: 28 - DNA, 220 bp, homo sapiens
cgtgcctgcgggagggcgccgagcatgtcgctggagtcgggcgcagaccgtgccctacctgccctccaactgggccaacaccggcttctcacttgtggtggccgcgtgctgcgagctcaccttgtggtcccggcaatgcaatgcgggcaagagggacaatttctgtggggtgagctagcgttgactgggaggagtaccgccgggggcatgttaggagact

SEQ ID NO: 29 - DNA, 500 bp, homo sapiens
gagccactctacagactttgatattcacatgtagcgtctgcatctgaaattgtttttacatctgtcccacctgcacccttcaccccaggctgttagtttcttgaggacaaggacttcatcattttcaaacattattggtcaaataaatgaagaaataggctgcatcctttctctttatcctttgacctcctctatcatcctgctgttatcttccagaaggagaagaaacagcttcacaggaaaagtagaggagattttcccattttggtgaaagtgccaaatcagaatgtgaaataggaattctgggctctgtaccaggcatttactcctatgctgttagctgatgttaaagagggtggatttcttttcccttaggtctcaccttctgtgccttcaggggaagttggttggaagtttgaatggtttgttgttgtcgtcattgttttgtattaaggagggctgtaatggaacgaatacaatggttattgatggagagtaag

SEQ ID NO: 30 - DNA, 298 bp, homo sapiens
aagaaaagcaacatagtggggtttctgtcaatctgtcctcggctgcccttctcatttgttgatgggaccttgaaagcaagcttgctaggtgccctctgtggctccagcctttaccggaagtgtggtgcatgtttttaacttcagggaagcggtatcctgtcactggggtatgggatgagcatggagaagaggcaccagccacgattccttcctaagcatctcctgttctgactgctcatgaattgaagaaactgacccttgtgttcaaaaaaaaaaggcaaagaggaagggtggcgga

SEQ ID NO: 31 - DNA, 970 bp, homo sapiens
gcgcgtgcccagactcccaaagtgctaggattacacagccctcgcccctcgggtcccttctgcccttgtgggagttgccagtcctcctatcctcccagtacaaagctggcttccacttctgcgggggctaccacatcagcgaggactgggtggtcaccgctgcccaccaggggtcaggtgaggcttgagcttgaggagttggggcccaccaggtgtcagggaatgacactcatcccccttcccaaccccaactacaccctgcaaccaacaggaaaagggtcggaaatgaactgcaggctggggaaccagcacccccatcccccggccacaggcagagccgcctctgcaggtgacacccaccccaggccgtgcaccccacctccacccttgcaggccacccagacggcagcttggggaaacctgggaggtcccgtaccctcactgtgcaggtggggaaatttagaccctgaaaaagggatgccctgagatcaccatgagattgaggggcaagcagggctcaccctgactggctcacttcccaggcacccccatgagcccaggcaccgcctgccaccctcactctccaggaagagccaccgcgtggtggccgggatcgtgtggtggccagggcgtctgaccttggctctcacccggaggccatccaggtgctgaggatggctaacgctaaggccacacagccagggagaggaggtggctcgtgacaccacgatgggacacacccacctctgggagaggagggtgactccgacagcccttgcctgccaggatggagcctggactctggagggcatcgtgtcctggagcagcaccagcacctcctgttgtcaccaggcgtggatgcccgcatcatgaaattcatgcctgggaaggcgtggaggccgagaccaccccccctaccccgcatctgtgtaaaacacaaataaagccattgagagcaactgcaaaaaaa

SEQ ID NO: 32-34 - skipped (3 entries)

SEQ ID NO: 35 - DNA, 1032 bp, homo sapiens
tagccatgaatgggataagcggagatcagggaactttctagccagggagaccaacaatgaacataaataagtaaatgatacagtatgtgaggtgacgaatgcgggggaggaaaactaaaactagaccaggtgaggtgaatcgggaacactggagggtggaggtggggcaggtgggttgaagggtgggcctcattgacagagtgacatttgggcaactattgaaggaagtgggtggaggaggtggcagccaggaggctatccagctaggaagagccttccaggcagaggaacattctagagcaaaggctctacagtgtggtctggccggggatgttgctggaacaacaagactggtgtggctgcctccaagtaagtgaaggtcaaagctgggtagaaggctgagggggcagggctgcgtacacacctcacacgcttcctgcctgagtgcctaggccttggtgggcaagcaggtcacaatcttatccaaggttgacctcacttttggactccaggccttctttggaaactgattcaactctgagtcaaggttttgcttcattcaacatggtcaagttaaattctgatatgcagcctactgctctctcctgggccaattcaaaagtggcttcattacccactgttaattacagtttcccgggcaaagtttcctcctaaaagaaagacaaggggcttttgctttgactctggctctttgcactttccccctcacagaccagagccccaggggaacaaatcctggttacccaagcagggaactcggtatatttaggggcttcaccgaaagggctgttggacagaggctgttttcagaggggcttgtgcaattgcagagacttccttgcatcccctagtgaataggggaaccattgctggtctcctctttggacctcttggcttagaccttttagtgggagtttcttggaagtgaggctgctggtcaacagcagaccttagtagaggggggttccactcagcattgcacaaggcacagggctttggcaggttggatct

SEQ ID NO: 36 - DNA, 1400 bp, homo sapiens
aagaaaagcaacatagtggggtttctgtcaatctgtcctcggctgcccttctcatttgttgatgggaccttgaaagcaagcttgctaggtgccctctgtggctccagcctttaccggaagtgtggtgcatgtttttaacttcagggaagcggtatcctgtcactggggtatgggatgagcatggagaagaggcaccagccacgattccttcctaagcatctcctgttctgactgctcatgaattgaagaaactgacccttgtgttcactctgcttcctctgattgtttctatcatggtttccaaaaattgccatgggaacatgtcaaagggccacttttggagactctgctccaggaagaactagtccccatagctcccctatcccccaccataccacagacatgctgtgacttagagaactacacaaacatccttgggacctagatgctggaggaatgacttaatttgatgcagaaactccatcacccaagggagtacctctcactgtaaacagtgctgtgcttgctctgaaggattaagcaagaagtcccaacagaagcaatgcaccagtcctgctatgggaatgaaactgcaaagcctaggagatgggtgaagtccctttctggagacctttaatgagtacttcaaagcactcgacacagatgcagcaaatagggcatcacacatacacaggcattaatactcacgggcatatagatgctgacatgtgtacactgacttacgcccttcccacagctacagataaggcctcgcaaagttggcctcagagacacatcaggaaccaaggtggaccagcaggtgccgagcctgtgtatctgcttggaggagacgttccaatgtgctgccttgttcagagatggtgtagttgcaagaaacagaaacccaccacaatttctcaggcaaaaagggagttaattataaggacataagagcacaaagttccagtgcaagagatacatccaggctgcacaagctccgggagtggggcctggcaagccaaaagaaaccaaagtttgtcttgccttctgttcctctttctgaagccacatagccttttatgactgtgtatctttgcatcgcttttgttttctttttatgtctctgaagccagcttttcctgttcactcatcccttgattaaatatggacattctagcttcacatcacttcctaattcagggaccaacagagactggttagcatgttgctatcccaacttccaaatggattaggggggtttgatttggcgggttgggggaagggggccctggttttggccagggggttgggggccatgtggggacataggccaaaggggctggagcctaatgggggcaggttttgacaacaaatggggtcatttcttgggacagacattc

SEQ ID NO: 37 - DNA, 366 bp, homo sapiens
tgtgcgtgcaatataccacaatataccatcatcctttctatttatgtggagactagttcaatcgatttttctgtcacctaagaatttacctaccccaggagcctgccttccacacatacattaatagacaccaaccagtaatgtcaaaaggaaaaattacaaacccagaaaattaaagtcattctgcacttgcccttggtttaacaggcatttcactcttggcacctttcctgtcctatcattaataagcatcttattgatacagtttatactccaaattctccaggcttgtgaaagtttcctcaggattgcttgaaaatgaaagtcctggccaggtgcgcagtggctcaggcctgtaatcccagc

SEQ ID NO: 38 - skipped

SEQ ID NO: 39 - protein, 130 aa, homo sapiens
VLPRRCLVFVVNTMDSSREPTLGRLDAAGFWQVWQRFDADEKGYIEEKELDAFFLHMLMKLGTDDTVMKANLHKVKQQFMTTQDASKDGRIRMKELAGMFLSEDENFLLLFRRENPLDKQRGVYADLAQI

SEQ ID NO: 40 - protein, 127 aa, homo sapiens
EHTSKLFHPNATIFRGILGSHKLLFHLVQICFHDRVISTQFHQHVEKESIEFLLFYVTFFIRIKALPDLPEASGVQAPQSRFPGAVHGVDDEDQAPARKDLDNFGKDSFTVFLDLLSCFFRYLISFE

SEQ ID NO: 41 - protein, 139 aa, homo sapiens
ELLRGRRAVAGVGRRPALPALQLGQHRLLTCGGPALRAHRVVPARQGGQDAQVLCRHLPAPGGVPPGHLRRLVQRYLRALLQVQLMHCWSLICSFHRVPSPSLSPSLGSAPGPQDPGGGAFCLPPPTSPAIQPLCSCPR

SEQ ID NO: 42 - protein, 133 aa, homo sapiens
SPLRPLLLALALASVPCAQGACPASADLKHSDGTRTCAKLYDKSDPYYENCCGGAELSLESGADLPYLPSNWANTASSLVVAPRCELTVWSRQGKAGKTHKFSAGTYPRLEEYRRGILGDWSNAISALYCRCS

SEQ ID NO: 43 - protein, 117 aa, homo sapiens
PRCARCCWPWPLPPCLAPRAPAPPPPTSSTRTGRALAPSSMTRATPTMRTAAGAPSCRWSRAQTCPTCPPTGPTPPPHLWWPRAASSPCGPGKARRARRTSSLPAPTRAWRSTAGAS

SEQ ID NO: 44 - protein, 160 aa, homo sapiens
RLDPDGSCESENIKVFDGTSSNGPLLGQVCSKNDYVPVFESSSSTLTFQIVTDSARIQRTVFVFYYFFSPNISIPNCGGYLDTLEGSFTSPNYPKPHPELAYCVWHIQVEKDYKIKLNFKEIFLEIDKQCKFDFLAIYDGPFNNFGLIGQVCGRVDCQLG

SEQ ID NO: 45 - protein, 73 aa, homo sapiens
RACGRAPSMSLESGADRALPALQLGQHRLLTCGGRVLRAHLVVPAMQCGQEGQFLWGELALTGRSTAGGMLGD

SEQ ID NO: 46 - protein, 78 aa, homo sapiens [sequence omitted]

SEQ ID NO: 47 - protein, 50 aa, homo sapiens
WDLESKLARCPLWLQPLPEVWCMFLTSGKRYPVTGVWDEHGEEAPATIPS

SEQ ID NO: 48 - protein, 70 aa, homo sapiens
EGIVAGASSPCSSHTPVTGYRFPEVKNMHHTSGKGWSHRGHLASLLSRSHQQMRRAAEDRLTETPLCCFS

SEQ ID NO: 49 - protein, 51 aa, homo sapiens [sequence omitted]

SEQ ID NO: 50 - protein, 161 aa, homo sapiens [sequence omitted]

SEQ ID NO: 51 - protein, 107 aa, homo sapiens [sequence omitted]

SEQ ID NO: 52 - protein, 118 aa, homo sapiens
QQEVLVLLQDTMPSRVQAPSWQARAVGVTLLSQRWVCPIVVSRATSSPWLCGLSVSHPQHLDGLRVRAKVRRPGHHTIPATTRWLFLESEGGRRCLGSWGCLGSEPVRVSPACPSISW

SEQ ID NO: 53-55 - skipped (3 entries)

SEQ ID NO: 56 - protein, 76 aa, homo sapiens
VPRPWWASRSQSYPRLTSLLDSRPSLETDSTLSQGFASFNMVKLNSDMQPTALSWANSKVASLPTVNYSFPGKVSS

SEQ ID NO: 57 - protein, 78 aa, homo sapiens [sequence omitted]

SEQ ID NO: 58 - protein, 136 aa, homo sapiens [sequence omitted]

SEQ ID NO: 59 - protein, 115 aa, homo sapiens [sequence omitted]

SEQ ID NO: 60 - protein, 70 aa, homo sapiens; identical to SEQ ID NO: 48

SEQ ID NO: 61 - protein, 95 aa, homo sapiens
LPFCLRNCGGFLFLATTPSLNKAAHWNVSSKQIHRLGTCWSTLVPDVSLRPTLRGLICSCGKGVSQCTHVSIYMPVSINACVCVMPYLLHLCRVL

SEQ ID NO: 62 - protein, 62 aa, homo sapiens
TPTSNVKRKNYKPRKLKSFCTCPWFNRHFTLGTFPVLSLISILLIQFILQILQACESFLRIA

SEQ ID NO: 63 - protein, 69 aa, homo sapiens [sequence omitted]

SEQ ID NO: 64-66 - skipped (3 entries)

SEQ ID NO: 67 - DNA, 1850 bp, homo sapiens
gagagccaattgacgtggtatttatttccatcgtcttgtaatgaaacacaccgacacttttttttgcaggcctgtgtgtcactttcaggcccttcctcatttattcataatatgtgtagccacgaggtagcatttcctttggcatcctggagaaacatgtctcacttagggttggacctgttagttaatagttctgcagcttctggtatttgtagtctgcccgttgatttacaaaatgcctcactgtgattgtcgctacagtcaccacattcagagctagaaccatgaaggaaaacagatgcacactgttgaaaggctggtttggagtttcttccgcatgtgtttcatgctgaaatcctgaattgccacttgcacttcgatcccttttcagacgaatgggtcctatgatggaatctgttttccatttatatgaagaaatgtctcgtttgcttctggagacacaaccttgattgcagcgagactggtggtcactgctatcacatatcaaaactttacactgcagatacacagagctcatacttctcaagaatttaaaggcattaaactggaatctcccatagtgtccaaataagggatacaccttacaagtttcatctcgactacatccactcttgattaggtcgtaggttggagatgcaaagtcagaggtgggagaggctctacaggtatcaagaaacaccaccaaatttggatctgaggtgtgcagactaacttgaacaaaaagagtttggttcaaatccacataatatggtgattcaagtatagtcttttcaaatgaattggattcaaaaagagccatgctggtgttatatttgcccagtgcattttgactttgtattacatcatcttctgttatgtatattatctccactgtagaattatgtcccatttcacacttcacaataatctggagttgtttctgacgggtgatcacttcagaagttgaggatgcagaaaaggtgattatattggtgtaagtaattgactgatcttctacctttctgattgtaccacatccattaagagggacagaaaattccacaacatttgataattttggtctgcaagttgggtcttttagttgcaagttattcccattagagttaaaagcctctaggtaggatttgcttataataactctcatcctgtcagaagagcaagttaaagatgtagtgttgatgttttctgcataaattgaggtgtaggaagcagaaaatccccggtaagaattggcataatctgtagacaacacgacagtcagagagtttgatgacgattcgaaggtgggagtcacacggccacagacttgtccaatcaggccagagttggtggaggggccatcatagatggcaagaaaatcaaatttgcactgtttgtctatttctaggaaaatctctttgaagtttagttttatcttgtaatctttctccacttgtatgtgccacacacaataagccagctcaggatgcggctttgggtaattggggctggtgaaggatccttccaaggtatccaggtaaccgccacagtttggaatagagatgttaggagagaagaagtagtagaagacaaagacagttctttgaattcttgctgagtcagtaactatttgaaacgtcaatgtactggatgatgattcaaatacaggaacatagtcgtttttactgcagacttgccctagcagaggcccattgctggaggttccgtcaaagactttaatgttttcactttcacagcttccatctggatcaagccttcagcctccgccattgtcactccgccaaacaggagagaattaagagggtcaatggcatgagccttcttacaag

SEQ ID NO: 68 - protein, 74 aa, homo sapiens
STPAVHHSCPPPPSHWHEASLAGQHCDRRRLPRQRRPDAPATSKCEPPTRKDYHNMFRVGSLGARTHVSDSGAL

SEQ ID NO: 69 - protein, 63 aa, homo sapiens
SNQRPRSTTPAHRPPATGTKHLWLGNTATGADYPDSAGLMLRRPASVNLLPGKITTTCSVWAP

SEQ ID NO: 70 - protein, 530 aa, homo sapiens
RLDPDGSCESENIKVFDGTSSNGPLLGQVCSKNDYVPVFESSSSTLTFQIVTDSARIQRTVFVFYYFFSPNISIPNCGGYLDTLEGSFTSPNYPKPHPELAYCVWHIQVEKDYKIKLNFKEIFLEIDKQCKFDFLAIYDGPSTNSGLIGQVCGRVTPTFESSSNSLTVVLSTDYANSYRGFSASYTSIYAENINTTSLTCSSDRMRVIISKSYLEAFNSNGNNLQLKDPTCRPKLSNVVEFSVPLNGCGTIRKVEDQSITYTNIITFSASSTSEVITRQKQLQIIVKCEMGHNSTVEIIYITEDDVIQSQNALGKYNTSMALFESNSFEKTILESPYYVDLNQTLFVQVSLHTSDPNLVVFLDTCRASPTSDFASPTYDLIKSGCSRDETCKVYPLFGHYGRFQFNAFKFLRSMSSVYLQCKVLICDSSDHQSRCNQGCVSRSKRDISSYKWKTDSIIGPIRLKRDRSASGNSGFQHETHAEETPNQPFNSVHLFSFMVLALNVVTVATITVRHFVNQRADYKYQKLQNY

SEQ ID NO: 71 - protein, 82 aa, homo sapiens [sequence omitted]

(19) United States
(12) Patent Application Publication
(10) Pub. No.: US 2004/0002471 A1
(43) Pub. Date: Jan. 1, 2004

(54) Human nucleic acid sequences which are overexpressed in normal pancreas tissue

(75) Inventors: Thomas Specht, Berlin (DE); Bernd Hinzmann, Berlin (DE); Armin Schmitt, Berlin (DE); Christian Pilarsky, Schonfeld (DE); Edgar Dahl, Potsdam (DE); Andre Rosenthal, Berlin (DE)

Correspondence Address: MILLEN, WHITE, ZELANO & BRANIGAN, P.C., 2200 CLARENDON BLVD., SUITE 1400, ARLINGTON, VA, 22201, US

(73) Assignee: Metagen Gesellschaft Fur Genomforschung MBH, Berlin (DE)

(21) Appl. No.: 10/330,051

(22) Filed: Dec. 30, 2002

Related U.S. Application Data
(62) Division of application No. 09/647,801, filed on Dec. 20, 2000, now abandoned.
Application No. 09/647,801 is a 371 of international application No. PCT/DE99/01096, filed on Apr. 8, 1999.

(30) Foreign Application Priority Data
Apr. 19, 1998 (DE) 198 18 598.7

(51) Int. Cl. A61K 48/00; C12Q 1/68; C07H 21/04; C12P 21/02; C12N 5/06; C12N 9/64

(52) U.S. Cl. 514/44; 435/6; 435/69.1; 435/320.1; 435/325; 435/226; 536/23.2

(57) ABSTRACT

Human nucleic acid sequences—mRNA, cDNA, genomic sequences—from normal pancreatic tissue, which code for gene products or portions thereof, and their use, are described. In addition, the polypeptides that can be obtained by way of the sequences and their use are described.

The invention relates to human nucleic acid sequences from normal pancreatic tissue, which code for gene products or portions thereof, their functional genes that code at least one bioactive polypeptide and their use.

In addition, the invention relates to the polypeptides that can be obtained by way of the sequences and their use.

One of the main causes of death is the pancreatic tumor, for control of which new therapies are necessary. Previously used therapies, such as, e.g., chemotherapy, hormone therapy or surgical removal of tumor tissue, frequently do not result in a complete cure.

The cancer phenomenon often goes along with overexpression or underexpression of certain genes in degenerated cells, it still being unclear whether these altered expression rates are the cause or the result of the malignant transformation. Identification of these genes would be an important step for development of new therapies against cancer. Spontaneous formation of cancer is often preceded by a host of mutations. They can have the most varied effects on the expression pattern in the affected tissue, such as, e.g., underexpression or overexpression, but also expression of shortened genes. Several such changes due to these mutation cascades can ultimately lead to malignant degeneration. The complexity of these relationships makes an experimental approach very difficult.

A database that consists of so-called ESTs is used to look for candidate genes, i.e., genes that compared to the tumor tissue are more strongly expressed-in normal tissue. ESTs (expressed sequence tags) are sequences of cDNAs, i.e., mRNAs transcribed in reverse, therefore molecules that reflect gene expression. The EST sequences are determined for normal and degenerated tissue. These databases are offered to some extent commercially by various companies. The ESTs of the LifeSeq database, which is used here, are generally between 150 and 350 nucleotides long. They represent a pattern that is unmistakable for a certain gene, although this gene is normally very much longer (>2000 nucleotides). By comparison of the expression patterns of normal and tumor tissue, ESTs can be identified that are important for tumor formation and proliferation. There is, however, the following problem: Since the EST sequences that are found can belong to different regions of an unknown gene due to different constructions of cDNA libraries, in this case a completely incorrect ratio of the occurrence of these ESTs in the respective tissue would arise. This would only be noticed when the complete gene is known and thus ESTs can be assigned to the same gene.

It has now been found that this error possibility can be reduced if all ESTs from the respective tissue type are assembled beforehand, before the expression patterns are compared to one another. Overlapping ESTs of the same gene were thus combined into longer sequences (see FIG. 1, FIG. 2a and FIG. 3). This lengthening and thus coverage of an essentially larger gene, region in each of the respective bases are intended to largely avoid the above-described error. Since there were no existing software products for this purpose, programs for assembling genomic sections were employed, which were used modified and to which our own programs were added. A flow chart of the assembly procedure is shown in FIGS. 2b1-2b4.

Nucleic acid sequences Seq. ID Nos. 2-37, and 67, which play a role as candidate genes in the pancreatic tumor, have now been found.

Nucleic acid sequences Seq. ID Nos. 14, 24, 25, 27-31, 35-37 and 67 are of special interest.

The invention thus relates to nucleic acid sequences that code a gene product or a portion thereof, comprising

a) a nucleic acid sequence selected from the group of nucleic acid sequences Seq. ID Nos. 14, 24, 25, 27-31, 35-37, and 67,

b) an allelic variation of the nucleic acid sequences named under a)

or

c) a nucleic acid sequence that is complementary to the nucleic acid sequences named under a) or b).

In addition, the invention relates to a nucleic acid sequence according to one of the sequences Seq. ID Nos. 14, 24, 25, 27-31, 35-37, 67 or a complementary or allelic variant thereof and the nucleic acid sequences thereof, which have 90% to 95% homology to a human nucleic acid sequence.

The invention also relates to nucleic acid sequences Seq. ID Nos. 2-37, and 67 which-are expressed elevated in normal pancreatic tissue.

The invention further relates to nucleic acid sequences comprising a portion of the above-mentioned nucleic acid sequences in such a sufficient amount that they hybridize with sequences Seq. ID Nos. 14, 24, 25, 27-31, 35-37, and 67.

The nucleic acid sequences according to the invention generally have a length of at least 50 to 4500 bp, preferably a length of at least 150 to 4000 bp, especially a length of 450 to 3500 bp.

With the partial sequences Seq. ID Nos. 2-37 and 67 according to the invention, expression cassettes can also be built using current process practice, whereby on the cassette at least one of the nucleic acid sequences according to the invention is combined with at least one control or regulatory sequence generally known to one skilled in the art, such as, e.g., a suitable promoter. The sequences according to the invention can be inserted in a sense or antisense orientation.

A large number of expression cassettes or vectors and promoters which can be used are known in the literature.

Expression cassettes or vectors are defined as:

1. bacterial, such as, e.g., phagescript, pBs, φX174, pBluescript SK, pBs KS, pNH8a, pNH16a, pNH18a, pNH46a (Stratagene), pTrc99A, pKK223-3, pKK233-3, pDR540, pRIT5 (Pharmacia), 2. eukaryotic, such as, e.g., pWLneo, pSV2cat, pOG44, pXT1, pSG (Stratagene), pSVK3, pBPV, pMSG, pSVL (Pharmacia).

A control or regulatory sequence is defined as suitable promoters. Here, two preferred vectors are the pKK232-8 and the PCM7 vector. In particular, the following promoters are intended: lacI, lacZ, T3, T7, gpt, lambda P_{R}, trc, CMV, HSV thymidine-kinase, SV40, LTRs from retrovirus and mouse metallothionein-I.

The DNA sequences located on the expression cassette can code a fusion protein which comprises a known protein and a bioactive polypeptide fragment.

The expression cassettes are likewise the subject matter of this invention.

The nucleic acid fragments according to the invention can be used to produce full-length genes. The genes that can be obtained are likewise the subject matter of this invention.

The invention also relates to the use of the nucleic acid sequences according to the invention and the gene fragments that can be obtained from use.

The nucleic acid sequences according to the invention can be moved with suitable vectors into host cells, in which as the heterologous part, the genetic information which is contained on the nucleic acid fragments and which is expressed is located.

The host cells containing the nucleic acid fragments are likewise the subject matter of this invention.

Suitable host cells are, e.g., prokaryotic cell systems such as E. coli or eukaryotic cell systems such as animal or human cells or yeasts.

The nucleic acid sequences according to the invention can be used in the sense or antisense form.

Production of polypeptides or their fragments is done by cultivation of the host cells according to current cultivation methods and subsequent isolation and purification of the peptides or fragments, likewise using current methods. The invention further relates to nucleic acid sequences, which code at least a partial sequence of a bioactive polypeptide.

This invention further relates to polypeptide partial sequences, so-called ORF (open-reading-frame)-peptides, according to the sequence protocols Seq. ID Nos. 39-63 and 68-71.

The invention further relates to the polypeptide sequences that have at least 80% homology, especially 90% homology to the polypeptide partial sequences of Seq. ID Nos. 39-63 and 68-71 according to the invention.

The invention also relates to antibodies that are directed against a polypeptide or a fragment thereof and that are coded by the nucleic acids of sequences Seq. ID Nos. 2-37 and 67.

Antibodies are defined especially as monoclonal antibodies.

The antibodies according to the invention can be identified by, i.a., a phage display process. These antibodies are also the subject matter of the invention.

The polypeptide partial sequences according to the invention can be used in a phase display process. The polypeptides that are identified with this process and that bind to the polypeptide partial sequences according to the invention are also the subject matter of the invention.

The nucleic acid sequences according to the invention can also be used in a phage display process.

The polypeptides of sequences Seq. ID Nos. 39-63 and 68-71 according to the invention can also be used as tools for finding active ingredients against pancreatic tumors, which is likewise the subject matter of this invention.

Likewise the subject matter of this invention is the use of nucleic acid sequences according to sequences Seq. ID Nos. 2-37 and 67 for expression of polypeptides, which can be used as tools for finding active ingredients against pancreatic tumors.

The invention also relates to the use of the founds polypeptide partial sequences Seq. ID Nos. 39-63 and 68-71 as pharmaceutical agents in the gene therapy for treatment of pancreatic tumors or for the production of a pharmaceutical agent for treatment of pancreatic tumors.

The invention also relates to pharmaceutical agents that contain at least one polypeptide partial sequence Seq. ID Nos. 39-63 and 68-71.

The nucleic acid sequences found according to the invention can also be genomic or mRNA sequences.

The invention also relates to genomic genes, their exon and intron structures and their splice variants that can be obtained from cDNAs of sequences Seq. ID Nos. 2-37 and 67, and their use together with suitable regulatory elements, such as suitable promoters and/or enhancers.

With the nucleic acids according to the invention (cDNA sequences) Seq. ID Nos. 2-37 and 67, genomic BAC, PAC and cosmid libraries are screened, and specifically human clones are isolated via complementary base pairing (hybridization). The BAC, PAC and cosmid clones isolated in this way are hybridized using fluorescence-in-situ hybridization on metaphase chromosomes, and the corresponding chromosome sections on which the corresponding genomic genes lie are identified. BAC, PAC and cosmid clones are sequenced in order to clarify the corresponding genomic genes in their complete structure (promoters, enhancers, silencers, exons and introns). BAC, PAC and cosmid clones can be used as independent molecules for gene transfer (see FIG. 5).

The invention also relates to BAC, PAC and cosmid clones containing functional genes and their chromosomal localization according to sequences Seq. ID Nos. 2-37 and 67, for use as vehicles for gene transfer.

Meanings of Technical Terms and Abbreviations

Nucleic acids=Nucleic acids in this invention are defined as: mRNA, partial cDNA, full-length cDNA and genomic genes (chromosomes)

ORF=Open Reading Frame, a defined sequence of amino acids which can be derived from the cDNA sequence

Contig=A set of DNA sequences that can be combined as a result of very great similarities into one sequence (consensus)

Singleton=A contig that contains only one sequence

Module=Domain of a protein with a defined sequence, which represents one structural unit and which occurs in various proteins

N=selectively the nucleotide A, T, G or C

X=selectively one of the 20 naturally occurring amino acids

Explanation of the Alignment Parameters

minimal initial match=minimal initial identity area

maximum pads per read=maximum number of insertions

maximum percent mismatch=maximum deviation in %

### EXPLANATION OF FIGURES

FIG. 1 shows the systematic gene search in the Incyte LifeSeq database

FIG. 2a shows the principle of EST assembling

FIGS. 2b1-2b4 show the entire principle of EST assembling

FIG. 3 shows the in-silico subtraction of gene expression in various tissues

FIG. 4a shows the determination of tissue-specific expression via electronic Northern

FIG. 4b shows the electronic Northern

FIG. 5 shows the isolation of genomic BAC and PAC clones.

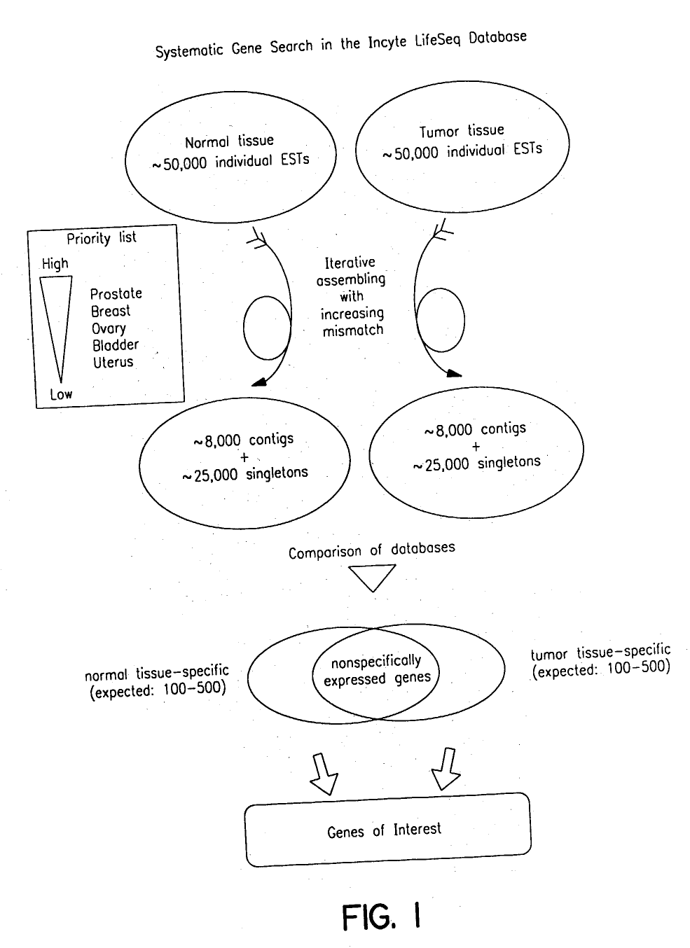

The following examples explain the production of the nucleic acid sequences according to the invention without limiting the invention to these examples and nucleic acid sequences.

### EXAMPLE 1

Search for Tumor-Related Candidate Genes

First, all ESTs of the corresponding tissue from the LifeSeq database (from October 1997) were extracted. They were then assembled by means of the GAP4 program of the Staden package with the parameters 0% mismatch, 8 pads per read and a minimal match of 20. The sequences (fails) not recorded in the GAP4 database were assembled first at 1% mismatch and then again at 2% mismatch with the database. Consensus sequences were computed from the contigs of the database that consisted of more than one sequence. The singletons of the database, which consisted of only one sequence, were re-assembled at 2% mismatch with the sequences not recorded in the GAP4 database. In turn, the consensus sequences were determined for the contigs. All other ESTs were re-assembled at 4% mismatch. The consensus sequences were extracted once again and finally assembled with the previous consensus sequences and the singletons and the sequences not recorded in the database at 4% mismatch. The consensus sequences were formed and used with the singletons and fails as the initial basis for tissue comparisons. This procedure ensured that among the parameters used, all sequences represented gene regions independent of one another.

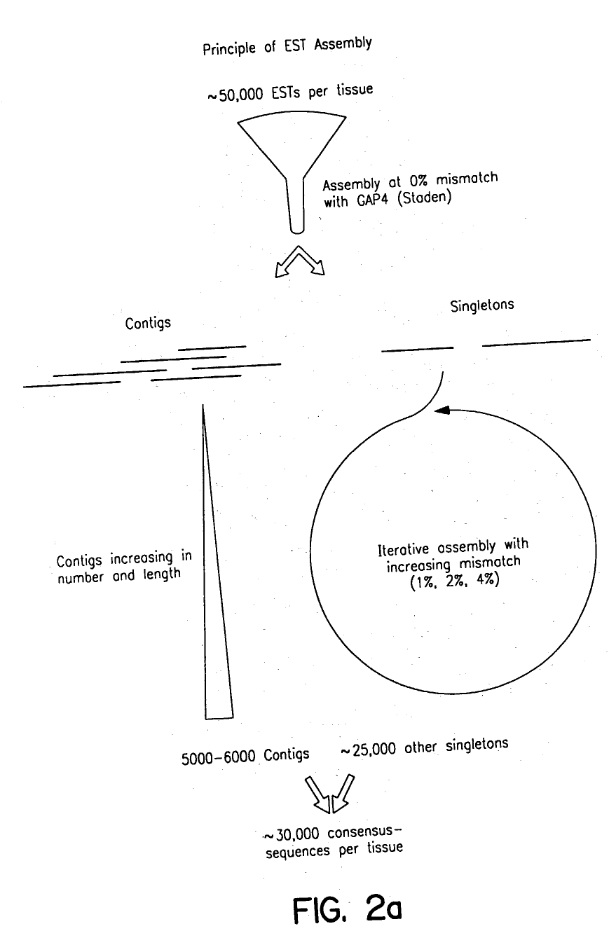

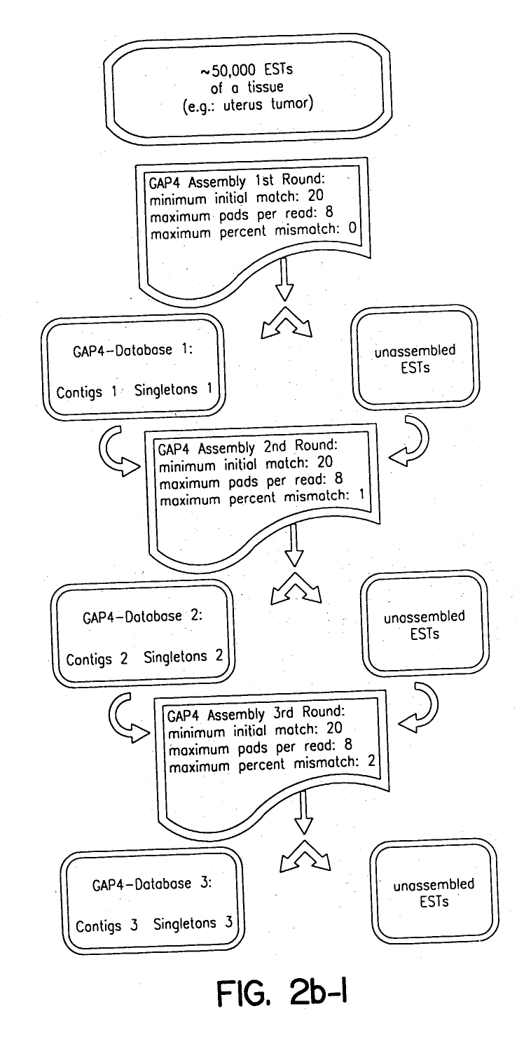

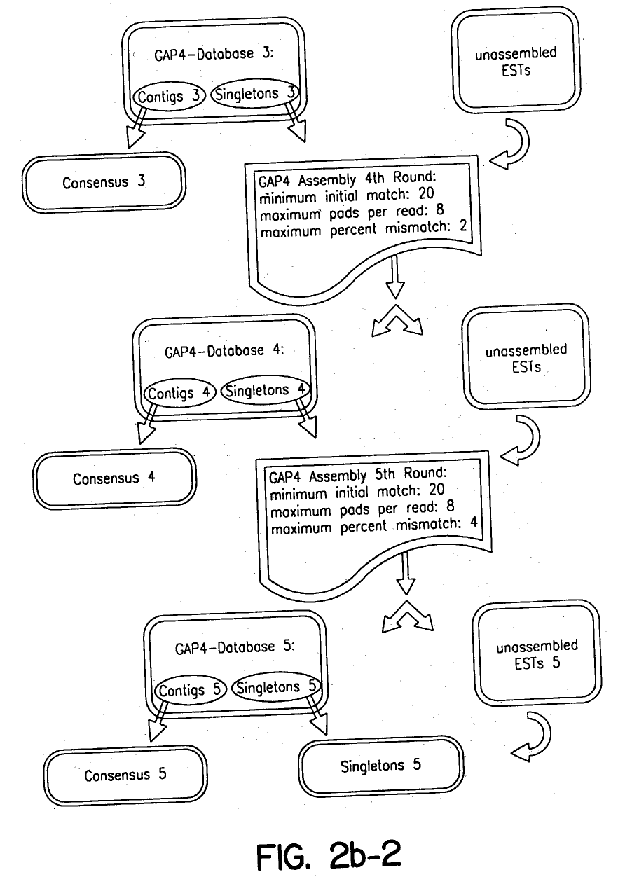

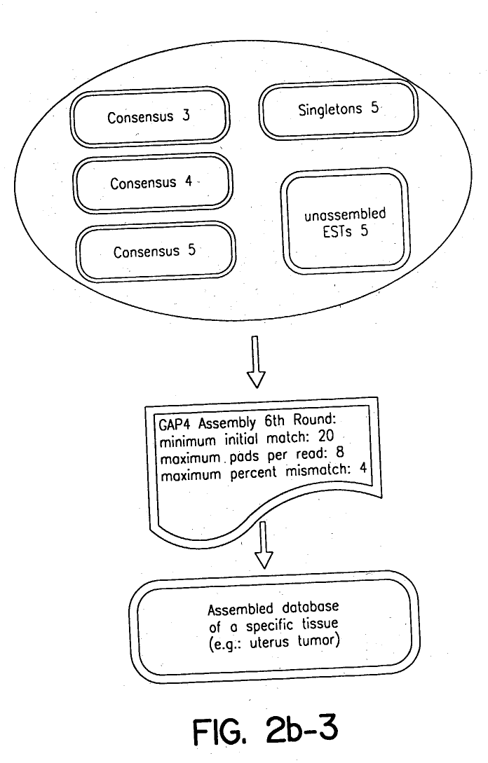

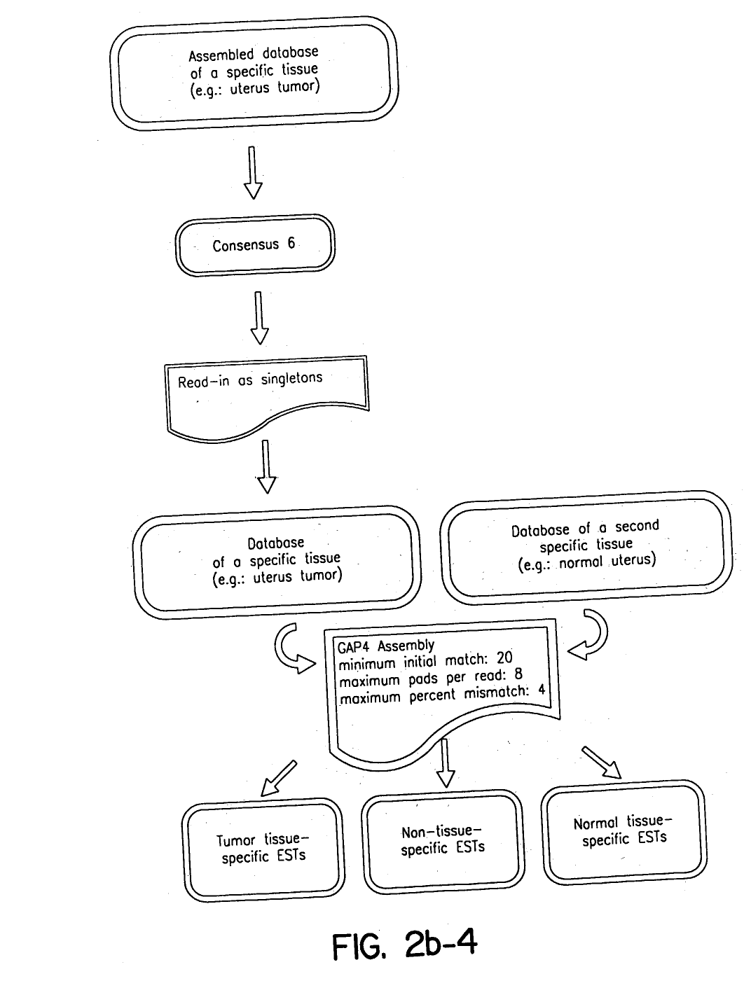

FIGS. 2b1-2b4 illustrate the lengthening of the normal pancreatic tissue ESTs.

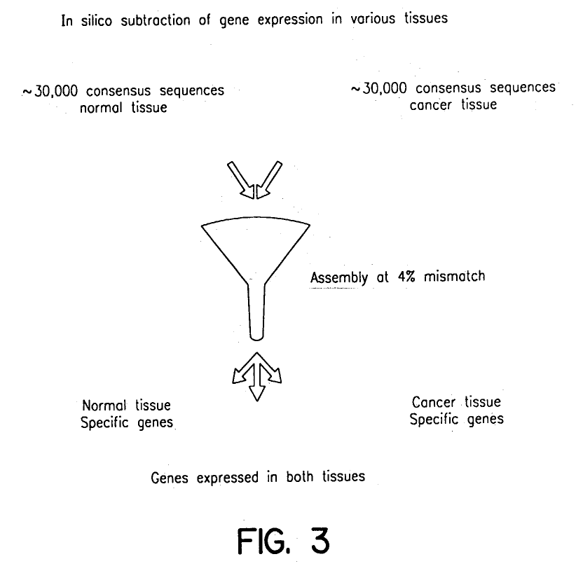

The sequences of the respective tissue assembled in this way were then compared to one another by means of the same program (FIG. 3). To do this, first all sequences of the first tissue were input into the database. (It was therefore important that they were independent of one another).

Then, all sequences of the second tissue were compared to all those of the first. The result was sequences that were specific to the first or the second tissue as well as those which occurred in both. In the latter, the ratio of the frequency of occurrence in the respective tissue was evaluated. All programs pertaining to the evaluation of the assembled sequences were themselves developed.

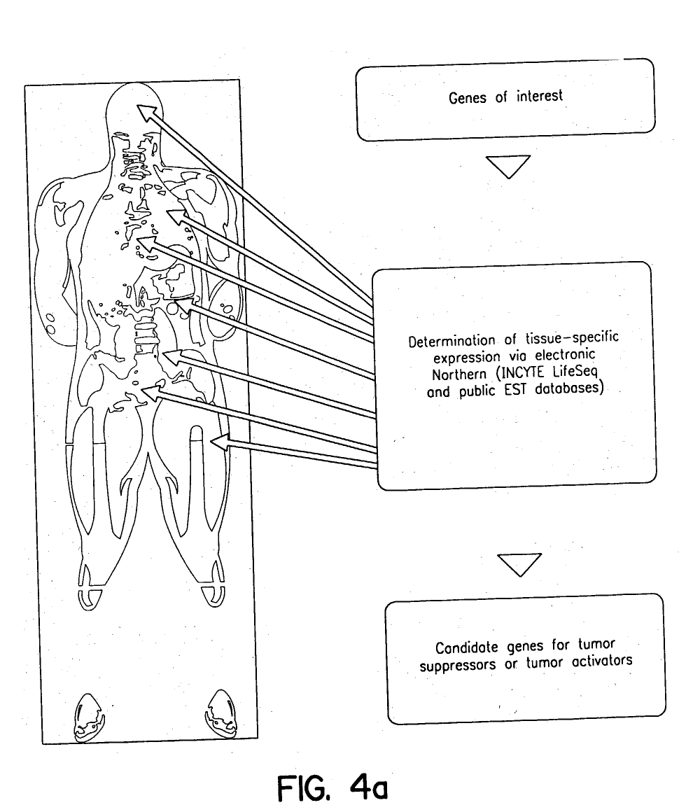

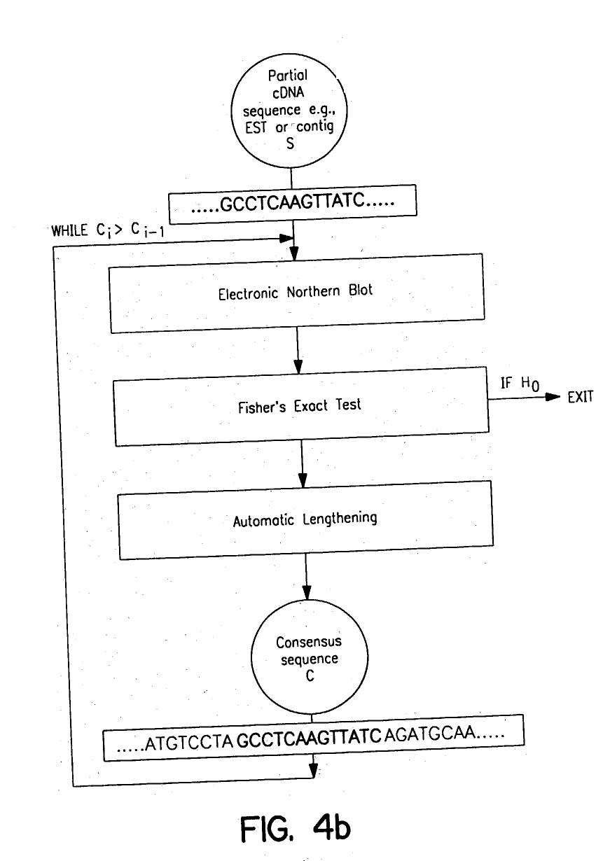

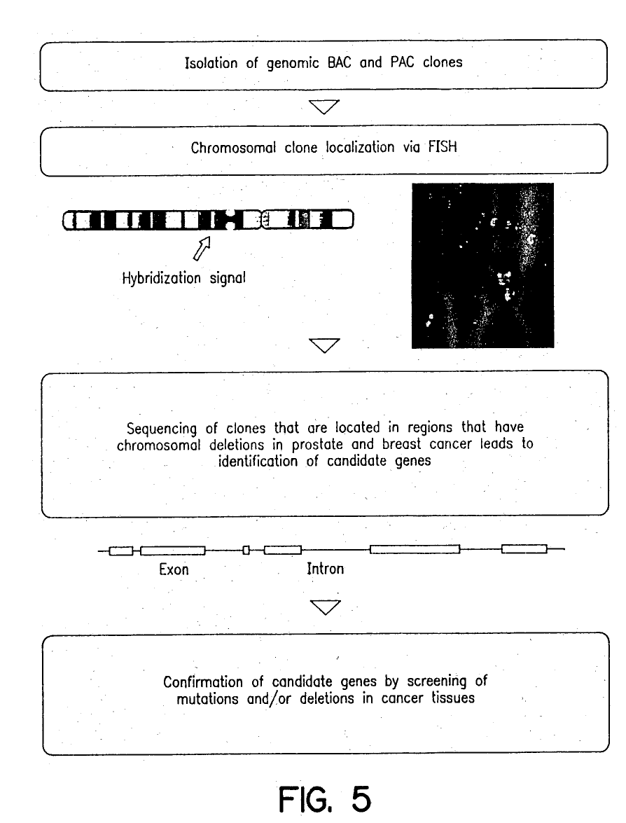

All sequences that occurred more than four times in respectively one of the compared tissues and all that occurred at least five times as often in one of the two tissues were further studied. These sequences were subjected to an electronic Northern (see Example 2.1), by which the distribution in all tumor and normal tissues was studied (see FIG. 4a and FIG. 4b). The relevant candidates were then lengthened using all Incyte ESTs and all ESTs of public databases (see Example 3). Then, the sequences and their translation into possible proteins were compared to all nucleotide and protein databases and were studied for possible regions that code for proteins.

### EXAMPLE 2

Algorithm for Identification and Lengthening of Partial cDNA Sequences with Altered Expression Pattern

An algorithm for finding overexpressed or underexpressed genes will be explained below. The individual steps are also summarized in a flow chart for the sake of clarity (see FIG. 4b).

2.1. Electronic Northern Blot

By means of a standard program for homology search, e.g., BLAST (Altschul, S. F.; Gish, W.; Miller, W.; Myers, E. W. and Lipman, D. J. (1990) J. Mol. Biol. 215, 403-410), BLAST2 (Altschul, S. F.; Madden, T. L.; Schaffer, A. A.; Zhang, J.; Zhang, Z.; Miller, W., and Lipman, D. J. (1997) Nucleic Acids Research 25 3389-3402) or FASTA (Pearson, W. R. and Lipman, D. J. (1988) Proc. Natl. Acad. Sci. USA 85 2444-2448), the homologous sequences in various EST libraries (private or public) arranged by tissues are determined for a partial DNA sequence S, e.g., an individual EST or a contig of ESTs. The (relative or absolute) tissue-specific occurrence frequencies of this partial sequence S which were determined in this way are called electronic Northern Blots.

2.1.1

Analogously to the procedure described under 2.1, the sequence Seq. ID No. 2 was found, which occurs 9× more frequently in normal pancreatic tissue than in tumor tissue.

The result is as follows:

Electronic Northern for SEQ. ID NO.: 2

| | NORMAL | TUMOR | Ratios | |
|---|---|---|---|---|
| | % frequency | % frequency | N/T | T/N |
| Bladder | 0.0000 | 0.0026 | 0.0000 | undef |
| Breast | 0.0000 | 0.0000 | undef | undef |
| Small intestine | 0.1502 | 0.0165 | 9.0831 | 0.1101 |
| Ovary | 0.0030 | 0.0026 | 1.1513 | 0.8686 |
| Endocrine tissue | 0.0000 | 0.0000 | undef | undef |
| Gastrointestinal | 0.0939 | 0.0093 | 10.1472 | 0.0985 |
| Brain | 0.0000 | 0.0010 | 0.0000 | undef |
| Hematopoietic | 0.0000 | 0.0000 | undef | undef |
| Skin | 0.0000 | 0.0000 | undef | undef |
| Hepatic | 0.0095 | 0.0194 | 0.4902 | 2.0400 |
| Heart | 0.0000 | 0.0000 | undef | undef |
| Testicles | 0.0000 | 0.0000 | undef | undef |
| Lung | 0.0000 | 0.0000 | undef | undef |
| Stomach-esophagus | 0.0000 | 0.1533 | 0.0000 | undef |
| Muscle-skeleton | 0.0000 | 0.0000 | undef | undef |
| Kidney | 0.0000 | 0.0068 | 0.0000 | undef |
| Pancreas | 2.7490 | 0.3037 | 9.0506 | 0.1105 |
| Penis | 0.0000 | 0.0000 | undef | undef |
| Prostate | 0.0000 | 0.0000 | undef | undef |
| Uterus-endometrium | 0.0000 | 0.0000 | undef | undef |
| Uterus-myometrium | 0.0000 | 0.0000 | undef | undef |
| Uterus-general | 0.0000 | 0.0000 | undef | undef |
| Breast hyperplasia | 0.0000 | | | |
| Prostate hyperplasia | 0.0000 | | | |
| Seminal vesicle | 0.0000 | | | |
| Sensory organs | 0.0000 | | | |
| White blood cells | 0.0000 | | | |
| Cervix | 0.0000 | | | |

| | | STANDARDIZED/SUBTRACTED | |
|---|---|---|---|
| | FETUS | LIBRARIES | |
| | % frequency | % frequency | |
| Development | 0.0000 | Breast | 0.0000 |
| Gastrointestinal | 0.0611 | Ovary_n | 0.0000 |
| Brain | 0.0000 | Ovary_t | 0.0000 |
| Hematopoietic | 0.0039 | Endocrine tissue | 0.0000 |
| Skin | 0.0000 | Fetal | 0.0000 |
| Hepatic | 0.0000 | Gastrointestinal | 0.1953 |
| Heart-blood vessels | 0.0036 | Hematopoietic | 0.0000 |
| Lung | 0.0000 | Skin-muscle | 0.0000 |
| Suprarenal gland | 0.0000 | Testicles | 0.0000 |
| Kidney | 0.0000 | Lung | 0.0000 |
| Placenta | 0.0000 | Nerves | 0.0000 |
| Prostate | 0.0000 | Prostate | 0.0000 |
| Sensory organs | 0.0000 | Sensory Organs | 0.0000 |
| | | Uterus_n | 0.0000 |

In an analogous procedure, the following Northerns were also found:

Electronic Northern for SEQ. ID NO.: 4

| | NORMAL | TUMOR | Ratios | |
|---|---|---|---|---|
| | % frequency | % frequency | N/T | T/N |
| Bladder | 0.0000 | 0.0000 | undef | undef |
| Breast | 0.0000 | 0.0000 | undef | undef |
| Small intestine | 0.0031 | 0.0000 | undef | 0.0000 |
| Ovary | 0.0000 | 0.0000 | undef | undef |
| Endocrine tissue | 0.0000 | 0.0000 | undef | undef |
| Gastrointestinal | 0.0038 | 0.0000 | undef | 0.0000 |
| Brain | 0.0000 | 0.0000 | undef | undef |
| Hematopoietic | 0.0000 | 0.0000 | undef | undef |
| Skin | 0.0000 | 0.0000 | undef | undef |
| Hepatic | 0.0000 | 0.0000 | undef | undef |
| Heart | 0.0000 | 0.0000 | undef | undef |
| Testicles | 0.0000 | 0.0000 | undef | undef |
| Lung | 0.0000 | 0.0000 | undef | undef |
| Stomach-esophagus | 0.0000 | 0.0000 | undef | undef |
| Muscle-skeleton | 0.0000 | 0.0000 | undef | undef |
| Kidney | 0.0027 | 0.0068 | 0.3965 | 2.5219 |
| Pancreas | 0.1355 | 0.0166 | 8.1767 | 0.1223 |
| Penis | 0.0000 | 0.0000 | undef | undef |
| Prostate | 0.0000 | 0.0021 | 0.0000 | undef |
| Uterus-endometrium | 0.0000 | 0.0000 | undef | undef |
| Uterus-myometrium | 0.0000 | 0.0000 | undef | undef |
| Uterus-general | 0.0000 | 0.0000 | undef | undef |
| Breast hyperplasia | 0.0000 | | | |
| Prostate hyperplasia | 0.0000 | | | |
| Seminal vesicle | 0.0000 | | | |
| Sensory organs | 0.0000 | | | |
| White blood cells | 0.0000 | | | |
| Cervix | 0.0000 | | | |

| | | STANDARDIZED/SUBTRACTED | |
|---|---|---|---|
| | FETUS | LIBRARIES | |
| | % frequency | % frequency | |
| Development | 0.0000 | Breast | 0.0000 |
| Gastrointestinal | 0.0028 | Ovary_n | 0.0000 |
| Brain | 0.0000 | Ovary_t | 0.0000 |
| Hematopoietic | 0.0000 | Endocrine tissue | 0.0000 |
| Skin | 0.0000 | Fetal | 0.0000 |
| Hepatic | 0.0000 | Gastrointestinal | 0.0000 |
| Heart-blood vessels | 0.0000 | Hematopoietic | 0.0000 |
| Lung | 0.0000 | Skin-muscle | 0.0000 |
| Suprarenal gland | 0.0000 | Testicles | 0.0000 |
| Kidney | 0.0000 | Lung | 0.0000 |
| Placenta | 0.0000 | Nerves | 0.0000 |
| Prostate | 0.0000 | Prostate | 0.0000 |
| Sensory organs | 0.0000 | Sensory Organs | 0.0000 |
| | | Uterus_n | 0.0000 |

Electronic Northern for SEQ. ID NO.: 6

| | NORMAL | TUMOR | Ratios | |
|---|---|---|---|---|
| | % frequency | % frequency | N/T | T/N |
| Bladder | 0.0000 | 0.0026 | 0.0000 | undef |
| Breast | 0.0000 | 0.0000 | undef | undef |
| Small intestine | 0.0031 | 0.0000 | undef | 0.0000 |
| Ovary | 0.0000 | 0.0000 | undef | undef |
| Endocrine tissue | 0.0000 | 0.0000 | undef | undef |
| Gastrointestinal | 0.0000 | 0.0000 | undef | undef |
| Brain | 0.0000 | 0.0000 | undef | undef |
| Hematopoietic | 0.0000 | 0.0000 | undef | undef |
| Skin | 0.0000 | 0.0000 | undef | undef |
| Hepatic | 0.0000 | 0.0000 | undef | undef |
| Heart | 0.0000 | 0.0000 | undef | undef |
| Testicles | 0.0000 | 0.0000 | undef | undef |
| Lung | 0.0062 | 0.0000 | undef | 0.0000 |
| Stomach-esophagus | 0.0000 | 0.0000 | undef | undef |
| Muscle-skeleton | 0.0000 | 0.0000 | undef | undef |
| Kidney | 0.0000 | 0.0000 | undef | undef |
| Pancreas | 0.8078 | 0.0166 | 48.7611 | 0.0205 |
| Penis | 0.0000 | 0.0000 | undef | undef |
| Prostate | 0.0000 | 0.0000 | undef | undef |
| Uterus-endometrium | 0.0000 | 0.0000 | undef | undef |
| Uterus-myometrium | 0.0000 | 0.0000 | undef | undef |
| Uterus-general | 0.0000 | 0.0000 | undef | undef |
| Breast hyperplasia | 0.0000 | | | |
| Prostate hyperplasia | 0.0000 | | | |
| Seminal vesicle | 0.0000 | | | |
| Sensory organs | 0.0000 | | | |
| White blood cells | 0.0000 | | | |
| Cervix | 0.0000 | | | |

| | | STANDARDIZED/SUBTRACTED | |
|---|---|---|---|
| | FETUS | LIBRARIES | |
| | % frequency | % frequency | |
| Development | 0.0000 | Breast | 0.0000 |
| Gastrointestinal | 0.0139 | Ovary_n | 0.0000 |
| Brain | 0.0000 | Ovary_t | 0.0000 |
| Hematopoietic | 0.0000 | Endocrine tissue | 0.0000 |
| Skin | 0.0000 | Fetal | 0.0000 |
| Hepatic | 0.0000 | Gastrointestinal | 0.0000 |
| Heart-blood vessels | 0.0000 | Hematopoietic | 0.0000 |
| Lung | 0.0000 | Skin-muscle | 0.0000 |
| Suprarenal gland | 0.0000 | Testicles | 0.0000 |
| Kidney | 0.0000 | Lung | 0.0000 |
| Placenta | 0.0000 | Nerves | 0.0000 |
| Prostate | 0.0000 | Prostate | 0.0000 |
| Sensory organs | 0.0000 | Sensory Organs | 0.0000 |
| | | Uterus_n | 0.0000 |

Electronic Northern for SEQ. ID NO.: 7

| | NORMAL | TUMOR | Ratios | |
|---|---|---|---|---|
| | % frequency | % frequency | N/T | T/N |
| Bladder | 0.0000 | 0.0000 | undef | undef |
| Breast | 0.0000 | 0.0000 | undef | undef |
| Small intestine | 0.0031 | 0.0000 | undef | 0.0000 |
| Ovary | 0.0000 | 0.0000 | undef | undef |
| Endocrine tissue | 0.0000 | 0.3310 | 0.0000 | undef |
| Gastrointestinal | 0.0019 | 0.0000 | undef | 0.0000 |
| Brain | 0.0089 | 0.0051 | 1.7279 | 0.5787 |
| Hematopoietic | 0.0000 | 0.0000 | undef | undef |
| Skin | 0.0000 | 0.0000 | undef | undef |
| Hepatic | 0.0000 | 0.0000 | undef | undef |
| Heart | 0.0000 | 0.0000 | undef | undef |
| Testicles | 0.0000 | 0.0000 | undef | undef |
| Lung | 0.0000 | 0.0000 | undef | undef |
| Stomach-esophagus | 0.0000 | 0.0000 | undef | undef |
| Muscle-skeleton | 0.0000 | 0.0000 | undef | undef |
| Kidney | 0.0000 | 0.0000 | undef | undef |
| Pancreas | 0.0991 | 0.0055 | 17.9489 | 0.0557 |
| Penis | 0.0000 | 0.0000 | undef | undef |
| Prostate | 0.0000 | 0.0000 | undef | undef |
| Uterus-endometrium | 0.0000 | 0.0000 | undef | undef |
| Uterus-myometrium | 0.0000 | 0.0000 | undef | undef |
| Uterus-general | 0.0000 | 0.0000 | undef | undef |
| Breast hyperplasia | 0.0000 | | | |
| Prostate hyperplasia | 0.0000 | | | |
| Seminal vesicle | 0.0000 | | | |
| Sensory organs | 0.0000 | | | |
| White blood cells | 0.0000 | | | |
| Cervix | 0.0000 | | | |

| | | STANDARDIZED/SUBTRACTED | |
|---|---|---|---|
| | FETUS | LIBRARIES | |
| | % frequency | % frequency | |
| Development | 0.0000 | Breast | 0.0000 |
| Gastrointestinal | 0.0389 | Ovary_n | 0.0000 |
| Brain | 0.0000 | Ovary_t | 0.0000 |
| Hematopoietic | 0.0000 | Endocrine tissue | 0.0000 |
| Skin | 0.0000 | Fetal | 0.0006 |
| Hepatic | 0.0000 | Gastrointestinal | 0.0000 |
| Heart-blood vessels | 0.0000 | Hematopoietic | 0.0000 |
| Lung | 0.0000 | Skin-muscle | 0.0000 |
| Suprarenal gland | 0.0000 | Testicles | 0.0000 |
| Kidney | 0.0000 | Lung | 0.0000 |
| Placenta | 0.0000 | Nerves | 0.0110 |
| Prostate | 0.0000 | Prostate | 0.0000 |
| Sensory organs | 0.0000 | Sensory Organs | 0.0000 |
| | | Uterus_n | 0.0000 |

Electronic Northern for SEQ. ID NO.: 9

| | NORMAL | TUMOR | Ratios | |
|---|---|---|---|---|
| | % frequency | % frequency | N/T | T/N |
| Bladder | 0.0000 | 0.0000 | undef | undef |
| Breast | 0.0000 | 0.0000 | undef | undef |
| Small intestine | 0.0000 | 0.0000 | undef | undef |
| Ovary | 0.0000 | 0.0000 | undef | undef |
| Endocrine tissue | 0.0000 | 0.0000 | undef | undef |
| Gastrointestinal | 0.0000 | 0.0000 | undef | undef |
| Brain | 0.0000 | 0.0000 | undef | undef |
| Hematopoietic | 0.0000 | 0.0000 | undef | undef |
| Skin | 0.0000 | 0.0000 | undef | undef |
| Hepatic | 0.0000 | 0.0000 | undef | undef |
| Heart | 0.0000 | 0.0000 | undef | undef |
| Testicles | 0.0000 | 0.0000 | undef | undef |
| Lung | 0.0000 | 0.0000 | undef | undef |
| Stomach-esophagus | 0.0000 | 0.0000 | undef | undef |
| Muscle-skeleton | 0.0000 | 0.0000 | undef | undef |
| Kidney | 0.0000 | 0.0000 | undef | undef |
| Pancreas | 0.1867 | 0.0000 | undef | 0.0000 |
| Penis | 0.0000 | 0.0000 | undef | undef |
| Prostate | 0.0000 | 0.0000 | undef | undef |
| Uterus-endometrium | 0.0000 | 0.0000 | undef | undef |
| Uterus-myometrium | 0.0000 | 0.0000 | undef | undef |
| Uterus-general | 0.0000 | 0.0000 | undef | undef |
| Breast hyperplasia | 0.0000 | | | |
| Prostate hyperplasia | 0.0000 | | | |
| Seminal vesicle | 0.0000 | | | |
| Sensory organs | 0.0000 | | | |
| White blood cells | 0.0000 | | | |
| Cervix | 0.0000 | | | |

| | | STANDARDIZED/SUBTRACTED | |
|---|---|---|---|
| | FETUS | LIBRARIES | |
| | % frequency | % frequency | |
| Development | 0.0000 | Breast | 0.0000 |
| Gastrointestinal | 0.0000 | Ovary_n | 0.0000 |
| Brain | 0.0000 | Ovary_t | 0.0000 |
| Hematopoietic | 0.0000 | Endocrine tissue | 0.0000 |
| Skin | 0.0000 | Fetal | 0.0000 |
| Hepatic | 0.0000 | Gastrointestinal | 0.0244 |
| Heart-blood vessels | 0.0000 | Hematopoietic | 0.0000 |
| Lung | 0.0000 | Skin-muscle | 0.0000 |
| Suprarenal gland | 0.0000 | Testicles | 0.0154 |
| Kidney | 0.0000 | Lung | 0.0000 |
| Placenta | 0.0000 | Nerves | 0.0000 |
| Prostate | 0.0000 | Prostate | 0.0000 |
| Sensory organs | 0.0000 | Sensory Organs | 0.0000 |
| | | Uterus_n | 0.0000 |

Electronic Northern for SEQ. ID NO.: 11

| | NORMAL | TUMOR | Ratios | |
|---|---|---|---|---|
| | % frequency | % frequency | N/T | T/N |
| Bladder | 0.0000 | 0.0128 | 0.0000 | undef |
| Breast | 0.0000 | 0.0000 | undef | undef |
| Small intestine | 0.0000 | 0.0000 | undef | undef |
| Ovary | 0.0060 | 0.0234 | 0.2558 | 3.9088 |
| Endocrine tissue | 0.0000 | 0.0000 | undef | undef |
| Gastrointestinal | 0.0096 | 0.0000 | undef | 0.0000 |
| Brain | 0.0000 | 0.0000 | undef | undef |
| Hematopoietic | 0.0000 | 0.0000 | undef | undef |
| Skin | 0.0000 | 0.0000 | undef | undef |
| Hepatic | 0.0190 | 0.0000 | undef | 0.0000 |
| Heart | 0.0011 | 0.0000 | undef | 0.0000 |
| Testicles | 0.0000 | 0.0000 | undef | undef |
| Lung | 0.0000 | 0.0020 | 0.0000 | undef |
| Stomach-esophagus | 0.0097 | 0.0077 | 1.2605 | 0.7933 |
| Muscle-skeleton | 0.0000 | 0.0000 | undef | undef |
| Kidney | 0.0000 | 0.0000 | undef | undef |
| Pancreas | 0.4180 | 0.0055 | 75.6843 | 0.0132 |
| Penis | 0.0000 | 0.0000 | undef | undef |
| Prostate | 0.0000 | 0.0000 | undef | undef |
| Uterus-endometrium | 0.0000 | 0.0000 | undef | undef |
| Uterus-myometrium | 0.0000 | 0.0000 | undef | undef |
| Uterus-general | 0.0000 | 0.0000 | undef | undef |
| Breast hyperplasia | 0.0000 | | | |
| Prostate hyperplasia | 0.0000 | | | |
| Seminal vesicle | 0.0000 | | | |
| Sensory organs | 0.0000 | | | |
| White blood cells | 0.0009 | | | |
| Cervix | 0.0000 | | | |

| | | STANDARDIZED/SUBTRACTED | |
|---|---|---|---|
| | FETUS | LIBRARIES | |
| | % frequency | % frequency | |
| Development | 0.0000 | Breast | 0.0000 |
| Gastrointestinal | 0.0944 | Ovary_n | 0.0000 |
| Brain | 0.0000 | Ovary_t | 0.0000 |
| Hematopoietic | 0.0000 | Endocrine tissue | 0.0000 |
| Skin | 0.0000 | Fetal | 0.0000 |
| Hepatic | 0.0000 | Gastrointestinal | 0.0000 |
| Heart-blood vessels | 0.0000 | Hematopoietic | 0.0000 |
| Lung | 0.0000 | Skin-muscle | 0.0000 |
| Suprarenal gland | 0.0000 | Testicles | 0.0000 |
| Kidney | 0.0000 | Lung | 0.0164 |
| Placenta | 0.0000 | Nerves | 0.0000 |
| Prostate | 0.0000 | Prostate | 0.0000 |
| Sensory organs | 0.0000 | Sensory Organs | 0.0000 |
| | | Uterus_n | 0.0000 |

Electronic Northern for SEQ. ID NO.: 12

| | NORMAL | TUMOR | Ratios | |
|---|---|---|---|---|
| | % frequency | % frequency | N/T | T/N |
| Bladder | 0.0000 | 0.0051 | 0.0000 | undef |
| Breast | 0.0000 | 0.0075 | 0.0000 | undef |
| Small intestine | 0.0061 | 0.0000 | undef | 0.0000 |
| Ovary | 0.0000 | 0.0000 | undef | undef |
| Endocrine tissue | 0.0000 | 0.0000 | undef | undef |
| Gastrointestinal | 0.0000 | 0.0000 | undef | undef |
| Brain | 0.0000 | 0.0041 | 0.0000 | undef |
| Hematopoietic | 0.0027 | 0.0000 | undef | 0.0000 |
| Skin | 0.0000 | 0.0000 | undef | undef |
| Hepatic | 0.0000 | 0.0000 | undef | undef |
| Heart | 0.0032 | 0.0000 | undef | 0.0000 |
| Testicles | 0.0000 | 0.0000 | undef | undef |
| Lung | 0.0000 | 0.0000 | undef | undef |
| Stomach-esophagus | 0.0000 | 0.0000 | undef | undef |
| Muscle-skeleton | 0.0000 | 0.0000 | undef | undef |
| Kidney | 0.0000 | 0.0000 | undef | undef |
| Pancreas | 3.8492 | 0.2706 | 14.2248 | 0.0703 |
| Penis | 0.0000 | 0.0000 | undef | undef |
| Prostate | 0.0000 | 0.0000 | undef | undef |
| Uterus-endometrium | 0.0000 | 0.0000 | undef | undef |
| Uterus-myometrium | 0.0000 | 0.0000 | undef | undef |
| Uterus-general | 0.0000 | 0.0000 | undef | undef |
| Breast hyperplasia | 0.0000 | | | |
| Prostate hyperplasia | 0.0000 | | | |
| Seminal vesicle | 0.0000 | | | |
| Sensory organs | 0.0000 | | | |
| White blood cells | 0.0009 | | | |
| Cervix | 0.0000 | | | |

| | | STANDARDIZED/SUBTRACTED | |
|---|---|---|---|
| | FETUS | LIBRARIES | |
| | % frequency | % frequency | |
| Development | 0.0000 | Breast | 0.0000 |
| Gastrointestinal | 0.2305 | Ovary_n | 0.0000 |
| Brain | 0.0000 | Ovary_t | 0.0000 |
| Hematopoietic | 0.0039 | Endocrine tissue | 0.0000 |
| Skin | 0.0000 | Fetal | 0.0000 |
| Hepatic | 0.0000 | Gastrointestinal | 0.4149 |
| Heart-blood vessels | 0.0000 | Hematopoietic | 0.0000 |
| Lung | 0.0000 | Skin-muscle | 0.0000 |
| Suprarenal gland | 0.0000 | Testicles | 0.0000 |
| Kidney | 0.0000 | Lung | 0.0000 |
| Placenta | 0.0000 | Nerves | 0.0000 |
| Prostate | 0.0000 | Prostate | 0.0000 |
| Sensory organs | 0.0000 | Sensory Organs | 0.0000 |
| | | Uterus_n | 0.0000 |

Electronic Northern for SEQ. ID NO.: 14

| | NORMAL | TUMOR | Ratios | |
|---|---|---|---|---|
| | % frequency | % frequency | N/T | T/N |
| Bladder | 0.0000 | 0.0000 | undef | undef |
| Breast | 0.0000 | 0.0000 | undef | undef |
| Small intestine | 0.0000 | 0.0000 | undef | undef |
| Ovary | 0.0000 | 0.0000 | undef | undef |
| Endocrine tissue | 0.0000 | 0.0000 | undef | undef |
| Gastrointestinal | 0.0000 | 0.0000 | undef | undef |
| Brain | 0.0000 | 0.0000 | undef | undef |
| Hematopoietic | 0.0000 | 0.0000 | undef | undef |
| Skin | 0.0000 | 0.0000 | undef | undef |
| Hepatic | 0.0000 | 0.0000 | undef | undef |
| Heart | 0.0000 | 0.0000 | undef | undef |
| Testicles | 0.0000 | 0.0000 | undef | undef |
| Lung | 0.0000 | 0.0000 | undef | undef |
| Stomach-esophagus | 0.0000 | 0.0000 | undef | undef |
| Muscle-skeleton | 0.0000 | 0.0000 | undef | undef |
| Kidney | 0.0000 | 0.0000 | undef | undef |
| Pancreas | 0.0182 | 0.0000 | undef | 0.0000 |
| Penis | 0.0000 | 0.0000 | undef | undef |
| Prostate | 0.0000 | 0.0000 | undef | undef |
| Uterus-endometrium | 0.0000 | 0.0000 | undef | undef |
| Uterus-myometrium | 0.0000 | 0.0000 | undef | undef |
| Uterus-general | 0.0000 | 0.0000 | undef | undef |
| Breast hyperplasia | 0.0000 | | | |
| Prostate hyperplasia | 0.0000 | | | |
| Seminal vesicle | 0.0000 | | | |
| Sensory organs | 0.0000 | | | |
| White blood cells | 0.0000 | | | |
| Cervix | 0.0000 | | | |

| | | STANDARDIZED/SUBTRACTED | |
|---|---|---|---|
| | FETUS | LIBRARIES | |
| | % frequency | % frequency | |
| Development | 0.0000 | Breast | 0.0000 |
| Gastrointestinal | 0.0028 | Ovary_n | 0.0000 |
| Brain | 0.0000 | Ovary_t | 0.0000 |
| Hematopoietic | 0.0000 | Endocrine tissue | 0.0000 |
| Skin | 0.0000 | Fetal | 0.0000 |
| Hepatic | 0.0000 | Gastrointestinal | 0.0000 |
| Heart-blood vessels | 0.0000 | Hematopoietic | 0.0000 |
| Lung | 0.0000 | Skin-muscle | 0.0000 |
| Suprarenal gland | 0.0000 | Testicles | 0.0000 |
| Kidney | 0.0000 | Lung | 0.0000 |
| Placenta | 0.0000 | Nerves | 0.0000 |
| Prostate | 0.0000 | Prostate | 0.0000 |
| Sensory organs | 0.0000 | Sensory Organs | 0.0000 |
| | | Uterus_n | 0.0000 |

Electronic Northern for SEQ. ID NO.: 15

| | NORMAL | TUMOR | Ratios | |
|---|---|---|---|---|
| | % frequency | % frequency | N/T | T/N |
| Bladder | 0.0000 | 0.0000 | undef | undef |
| Breast | 0.0000 | 0.0038 | 0.0000 | undef |
| Small intestine | 0.0061 | 0.0000 | undef | 0.0000 |
| Ovary | 0.0000 | 0.0000 | undef | undef |
| Endocrine tissue | 0.0000 | 0.0000 | undef | undef |
| Gastrointestinal | 0.0000 | 0.0000 | undef | undef |
| Brain | 0.0000 | 0.0051 | 0.0000 | undef |
| Hematopoietic | 0.0040 | 0.0000 | undef | 0.0000 |
| Skin | 0.0000 | 0.0000 | undef | undef |
| Hepatic | 0.0000 | 0.0000 | undef | undef |
| Heart | 0.0011 | 0.0000 | undef | 0.0000 |
| Testicles | 0.0000 | 0.0000 | undef | undef |
| Lung | 0.0000 | 0.0000 | undef | undef |
| Stomach-esophagus | 0.0000 | 0.0000 | undef | undef |
| Muscle-skeleton | 0.0000 | 0.0000 | undef | undef |
| Kidney | 0.0000 | 0.0000 | undef | undef |
| Pancreas | 2.8861 | 0.2872 | 10.0502 | 0.0995 |
| Penis | 0.0000 | 0.0000 | undef | undef |
| Prostate | 0.0000 | 0.0000 | undef | undef |
| Uterus-endometrium | 0.0000 | 0.0000 | undef | undef |
| Uterus-myometrium | 0.0000 | 0.0000 | undef | undef |
| Uterus-general | 0.0000 | 0.0000 | undef | undef |
| Breast hyperplasia | 0.0000 | | | |
| Prostate hyperplasia | 0.0000 | | | |
| Seminal vesicle | 0.0000 | | | |
| Sensory organs | 0.0000 | | | |
| White blood cells | 0.0009 | | | |
| Cervix | 0.0000 | | | |

| | | STANDARDIZED/SUBTRACTED | |
|---|---|---|---|
| | FETUS | LIBRARIES | |
| | % frequency | % frequency | |
| Development | 0.0000 | Breast | 0.0000 |
| Gastrointestinal | 0.2777 | Ovary_n | 0.0000 |
| Brain | 0.0000 | Ovary_t | 0.0000 |
| Hematopoietic | 0.0039 | Endocrine tissue | 0.0000 |
| Skin | 0.0000 | Fetal | 0.0000 |
| Hepatic | 0.0000 | Gastrointestinal | 0.0854 |
| Heart-blood vessels | 0.0000 | Hematopoietic | 0.0000 |
| Lung | 0.0000 | Skin-muscle | 0.0000 |
| Suprarenal gland | 0.0000 | Testicles | 0.0000 |
| Kidney | 0.0000 | Lung | 0.0000 |
| Placenta | 0.0000 | Nerves | 0.0000 |
| Prostate | 0.0000 | Prostate | 0.0000 |
| Sensory organs | 0.0000 | Sensory Organs | 0.0000 |
| | | Uterus_n | 0.0000 |

Electronic Northern for SEQ. ID NO.: 16

| | NORMAL | TUMOR | Ratios | |
|---|---|---|---|---|
| | % frequency | % frequency | N/T | T/N |
| Bladder | 0.0000 | 0.0026 | 0.0000 | undef |
| Breast | 0.0000 | 0.0000 | undef | undef |
| Small intestine | 0.0061 | 0.0000 | undef | 0.0000 |
| Ovary | 0.0000 | 0.0000 | undef | undef |
| Endocrine tissue | 0.0000 | 0.0000 | undef | undef |
| Gastrointestinal | 0.0000 | 0.0000 | undef | undef |
| Brain | 0.0000 | 0.0051 | 0.0000 | undef |
| Hematopoietic | 0.0000 | 0.0000 | undef | undef |
| Skin | 0.0000 | 0.0000 | undef | undef |
| Hepatic | 0.0000 | 0.0000 | undef | undef |
| Heart | 0.0011 | 0.0000 | undef | 0.0000 |
| Testicles | 0.0000 | 0.0000 | undef | undef |
| Lung | 0.0010 | 0.0000 | undef | 0.0000 |
| Stomach-esophagus | 0.0000 | 0.0000 | undef | undef |
| Muscle-skeleton | 0.0000 | 0.0000 | undef | undef |
| Kidney | 0.0000 | 0.0068 | 0.0000 | undef |
| Pancreas | 1.5232 | 0.0110 | 137.9070 | 0.0073 |
| Penis | 0.0000 | 0.0000 | undef | undef |
| Prostate | 0.0000 | 0.0000 | undef | undef |
| Uterus-endometrium | 0.0000 | 0.0000 | undef | undef |
| Uterus-myometrium | 0.0000 | 0.0000 | undef | undef |
| Uterus-general | 0.0000 | 0.0000 | undef | undef |
| Breast hyperplasia | 0.0000 | | | |
| Prostate hyperplasia | 0.0000 | | | |
| Seminal vesicle | 0.0000 | | | |
| Sensory organs | 0.0000 | | | |
| White blood cells | 0.0000 | | | |
| Cervix | 0.0000 | | | |

| | | STANDARDIZED/SUBTRACTED | |
|---|---|---|---|
| | FETUS | LIBRARIES | |
| | % frequency | % frequency | |
| Development | 0.0000 | Breast | 0.0000 |
| Gastrointestinal | 0.6386 | Ovary_n | 0.0000 |
| Brain | 0.0000 | Ovary_t | 0.0000 |
| Hematopoietic | 0.0197 | Endocrine tissue | 0.0000 |
| Skin | 0.0000 | Fetal | 0.0000 |
| Hepatic | 0.0000 | Gastrointestinal | 0.0000 |
| Heart-blood vessels | 0.0000 | Hematopoietic | 0.0000 |
| Lung | 0.0000 | Skin-muscle | 0.0000 |
| Suprarenal gland | 0.0000 | Testicles | 0.0000 |
| Kidney | 0.0000 | Lung | 0.0000 |
| Placenta | 0.0000 | Nerves | 0.0000 |
| Prostate | 0.0000 | Prostate | 0.0000 |
| Sensory organs | 0.0000 | Sensory Organs | 0.0000 |
| | | Uterus_n | 0.0000 |

Electronic Northern for SEQ. ID NO.: 17

| | NORMAL | TUMOR | Ratios | |
|---|---|---|---|---|
| | % frequency | % frequency | N/T | T/N |
| Bladder | 0.0000 | 0.0051 | 0.0000 | undef |
| Breast | 0.0000 | 0.0056 | 0.0000 | undef |
| Small intestine | 0.0000 | 0.0000 | undef | undef |
| Ovary | 0.0000 | 0.0026 | 0.0000 | undef |
| Endocrine tissue | 0.0000 | 0.0000 | undef | undef |
| Gastrointestinal | 0.0000 | 0.0000 | undef | undef |
| Brain | 0.0000 | 0.0041 | 0.0000 | undef |
| Hematopoietic | 0.0027 | 0.0000 | undef | 0.0000 |
| Skin | 0.0000 | 0.0000 | undef | undef |
| Hepatic | 0.0000 | 0.0000 | undef | undef |
| Heart | 0.0000 | 0.0000 | undef | undef |
| Testicles | 0.0000 | 0.0000 | undef | undef |
| Lung | 0.0010 | 0.0000 | undef | 0.0000 |
| Stomach-esophagus | 0.0000 | 0.0000 | undef | undef |
| Muscle-skeleton | 0.0000 | 0.0000 | undef | undef |
| Kidney | 0.0000 | 0.0000 | undef | undef |
| Pancreas | 0.9218 | 0.0387 | 23.8463 | 0.0419 |
| Penis | 0.0000 | 0.0000 | undef | undef |
| Prostate | 0.0000 | 0.0000 | undef | undef |
| Uterus-endometrium | 0.0000 | 0.0000 | undef | undef |
| Uterus-myometrium | 0.0000 | 0.0000 | undef | undef |
| Uterus-general | 0.0000 | 0.0000 | undef | undef |
| Breast hyperplasia | 0.0000 | | | |
| Prostate hyperplasia | 0.0000 | | | |
| Seminal vesicle | 0.0000 | | | |
| Sensory organs | 0.0000 | | | |
| White blood cells | 0.0000 | | | |
| Cervix | 0.0000 | | | |

| | | STANDARDIZED/SUBTRACTED | |
|---|---|---|---|
| | FETUS | LIBRARIES | |
| | % frequency | % frequency | |
| Development | 0.0000 | Breast | 0.0000 |
| Gastrointestinal | 0.1111 | Ovary_n | 0.0000 |
| Brain | 0.0000 | Ovary_t | 0.0000 |
| Hematopoietic | 0.0157 | Endocrine tissue | 0.0000 |
| Skin | 0.0000 | Fetal | 0.0000 |
| Hepatic | 0.0000 | Gastrointestinal | 0.0000 |
| Heart-blood vessels | 0.0000 | Hematopoietic | 0.0000 |
| Lung | 0.0000 | Skin-muscle | 0.0000 |
| Suprarenal gland | 0.0000 | Testicles | 0.0000 |
| Kidney | 0.0000 | Lung | 0.0000 |
| Placenta | 0.0000 | Nerves | 0.0000 |
| Prostate | 0.0000 | Prostate | 0.0000 |
| Sensory organs | 0.0000 | Sensory Organs | 0.0000 |
| | | Uterus_n | 0.0000 |

Electronic Northern for SEQ. ID NO.: 18

| | NORMAL | TUMOR | Ratios | |
|---|---|---|---|---|
| | % frequency | % frequency | N/T | T/N |
| Bladder | 0.0000 | 0.0026 | 0.0000 | undef |
| Breast | 0.0000 | 0.0000 | undef | undef |
| Small intestine | 0.0000 | 0.0000 | undef | undef |
| Ovary | 0.0030 | 0.0000 | undef | 0.0000 |
| Endocrine tissue | 0.0000 | 0.0000 | undef | undef |
| Gastrointestinal | 0.0000 | 0.0000 | undef | undef |
| Brain | 0.0000 | 0.0010 | 0.0000 | undef |
| Hematopoietic | 0.0000 | 0.0000 | undef | undef |
| Skin | 0.0000 | 0.0000 | undef | undef |
| Hepatic | 0.0000 | 0.0000 | undef | undef |
| Heart | 0.0000 | 0.0000 | undef | undef |
| Testicles | 0.0000 | 0.0000 | undef | undef |
| Lung | 0.0000 | 0.0000 | undef | undef |
| Stomach-esophagus | 0.0000 | 0.0000 | undef | undef |
| Muscle-skeleton | 0.0000 | 0.0000 | undef | undef |
| Kidney | 0.0054 | 0.0000 | undef | 0.0000 |
| Pancreas | 0.4659 | 0.0221 | 21.0899 | 0.0474 |
| Penis | 0.0000 | 0.0000 | undef | undef |
| Prostate | 0.0000 | 0.0000 | undef | undef |
| Uterus-endometrium | 0.0000 | 0.0000 | undef | undef |
| Uterus-myometrium | 0.0000 | 0.0000 | undef | undef |
| Uterus-general | 0.0000 | 0.0000 | undef | undef |
| Breast hyperplasia | 0.0000 | | | |
| Prostate hyperplasia | 0.0030 | | | |
| Seminal vesicle | 0.0000 | | | |
| Sensory organs | 0.0000 | | | |
| White blood cells | 0.0000 | | | |
| Cervix | 0.0000 | | | |

| | | STANDARDIZED/SUBTRACTED | |
|---|---|---|---|
| | FETUS | LIBRARIES | |
| | % frequency | % frequency | |
| Development | 0.0000 | Breast | 0.0000 |
| Gastrointestinal | 0.0305 | Ovary_n | 0.0000 |
| Brain | 0.0000 | Ovary_t | 0.0000 |
| Hematopoietic | 0.0039 | Endocrine tissue | 0.0000 |
| Skin | 0.0000 | Fetal | 0.0000 |
| Hepatic | 0.0000 | Gastrointestinal | 0.0000 |
| Heart-blood vessels | 0.0000 | Hematopoietic | 0.0000 |
| Lung | 0.0000 | Skin-muscle | 0.0000 |
| Suprarenal gland | 0.0000 | Testicles | 0.0000 |
| Kidney | 0.0000 | Lung | 0.0000 |
| Placenta | 0.0000 | Nerves | 0.0000 |
| Prostate | 0.0000 | Prostate | 0.0000 |
| Sensory organs | 0.0000 | Sensory Organs | 0.0000 |
| | | Uterus_n | 0.0000 |

Electronic Northern for SEQ. ID NO.: 19

| | NORMAL | TUMOR | Ratios | |
|---|---|---|---|---|
| | % frequency | % frequency | N/T | T/N |
| Bladder | 0.0000 | 0.0000 | undef | undef |
| Breast | 0.0000 | 0.0000 | undef | undef |
| Small intestine | 0.0000 | 0.0000 | undef | undef |
| Ovary | 0.0000 | 0.0000 | undef | undef |
| Endocrine tissue | 0.0000 | 0.0000 | undef | undef |
| Gastrointestinal | 0.0000 | 0.0000 | undef | undef |
| Brain | 0.0000 | 0.0000 | undef | undef |
| Hematopoietic | 0.0000 | 0.0000 | undef | undef |
| Skin | 0.0000 | 0.0000 | undef | undef |
| Hepatic | 0.0000 | 0.0000 | undef | undef |
| Heart | 0.0000 | 0.0000 | undef | undef |
| Testicles | 0.0000 | 0.0000 | undef | undef |
| Lung | 0.0000 | 0.0000 | undef | undef |
| Stomach-esophagus | 0.0000 | 0.0000 | undef | undef |
| Muscle-skeleton | 0.0000 | 0.0000 | undef | undef |
| Kidney | 0.0000 | 0.0000 | undef | undef |
| Pancreas | 0.0314 | 0.0000 | undef | 0.0000 |
| Penis | 0.0000 | 0.0000 | undef | undef |
| Prostate | 0.0000 | 0.0000 | undef | undef |
| Uterus-endometrium | 0.0000 | 0.0000 | undef | undef |
| Uterus-myometrium | 0.0000 | 0.0000 | undef | undef |
| Uterus-general | 0.0000 | 0.0000 | undef | undef |
| Breast hyperplasia | 0.0000 | | | |
| Prostate hyperplasia | 0.0000 | | | |
| Seminal vesicle | 0.0000 | | | |
| Sensory organs | 0.0000 | | | |
| White blood cells | 0.0000 | | | |
| Cervix | 0.0000 | | | |

| | | STANDARDIZED/SUBTRACTED | |
|---|---|---|---|
| | FETUS | LIBRARIES | |
| | % frequency | % frequency | |
| Development | 0.0000 | Breast | 0.0000 |
| Gastrointestinal | 0.0000 | Ovary_n | 0.0000 |
| Brain | 0.0000 | Ovary_t | 0.0000 |
| Hematopoietic | 0.0000 | Endocrine tissue | 0.0000 |
| Skin | 0.0000 | Fetal | 0.0000 |
| Hepatic | 0.0000 | Gastrointestinal | 0.0366 |
| Heart-blood vessels | 0.0000 | Hematopoietic | 0.0000 |
| Lung | 0.0000 | Skin-muscle | 0.0000 |
| Suprarenal gland | 0.0000 | Testicles | 0.0000 |
| Kidney | 0.0000 | Lung | 0.0000 |
| Placenta | 0.0000 | Nerves | 0.0000 |
| Prostate | 0.0000 | Prostate | 0.0000 |
| Sensory organs | 0.0000 | Sensory Organs | 0.0000 |
| | | Uterus_n | 0.0000 |

Electronic Northern for SEQ. ID NO.: 21

| | NORMAL | TUMOR | Ratios | |
|---|---|---|---|---|
| | % frequency | % frequency | N/T | T/N |
| Bladder | 0.0000 | 0.0026 | 0.0000 | undef |
| Breast | 0.0000 | 0.0000 | undef | undef |
| Small intestine | 0.0000 | 0.0000 | undef | undef |
| Ovary | 0.0000 | 0.0000 | undef | undef |
| Endocrine tissue | 0.0000 | 0.0000 | undef | undef |
| Gastrointestinal | 0.0000 | 0.0000 | undef | undef |
| Brain | 0.0000 | 0.0000 | undef | undef |
| Hematopoietic | 0.0000 | 0.0000 | undef | undef |
| Skin | 0.0000 | 0.0000 | undef | undef |
| Hepatic | 0.0000 | 0.0000 | undef | undef |
| Heart | 0.0000 | 0.0000 | undef | undef |
| Testicles | 0.0000 | 0.0000 | undef | undef |
| Lung | 0.0000 | 0.0000 | undef | undef |
| Stomach-esophagus | 0.0000 | 0.0000 | undef | undef |
| Muscle-skeleton | 0.0000 | 0.0000 | undef | undef |
| Kidney | 0.0000 | 0.0000 | undef | undef |
| Pancreas | 0.1586 | 0.0000 | undef | 0.0000 |
| Penis | 0.0000 | 0.0000 | undef | undef |
| Prostate | 0.0000 | 0.0000 | undef | undef |
| Uterus-endometrium | 0.0000 | 0.0000 | undef | undef |
| Uterus-myometrium | 0.0000 | 0.0000 | undef | undef |
| Uterus-general | 0.0000 | 0.0000 | undef | undef |
| Breast hyperplasia | 0.0000 | | | |
| Prostate hyperplasia | 0.0000 | | | |
| Seminal vesicle | 0.0000 | | | |
| Sensory organs | 0.0000 | | | |
| White blood cells | 0.0000 | | | |
| Cervix | 0.0000 | | | |

| | | STANDARDIZED/SUBTRACTED | |
|---|---|---|---|
| | FETUS | LIBRARIES | |
| | % frequency | % frequency | |
| Development | 0.0000 | Breast | 0.0000 |
| Gastrointestinal | 0.0000 | Ovary_n | 0.0000 |
| Brain | 0.0000 | Ovary_t | 0.0000 |
| Hematopoietic | 0.0000 | Endocrine tissue | 0.0000 |
| Skin | 0.0000 | Fetal | 0.0000 |
| Hepatic | 0.0000 | Gastrointestinal | 0.0000 |
| Heart-blood vessels | 0.0000 | Hematopoietic | 0.0057 |
| Lung | 0.0000 | Skin-muscle | 0.0000 |
| Suprarenal gland | 0.0000 | Testicles | 0.0000 |
| Kidney | 0.0000 | Lung | 0.0000 |
| Placenta | 0.0000 | Nerves | 0.0000 |
| Prostate | 0.0000 | Prostate | 0.0000 |
| Sensory organs | 0.0000 | Sensory Organs | 0.0000 |
| | | Uterus_n | 0.0000 |

Electronic Northern for SEQ. ID NO.: 24

| | NORMAL | TUMOR | Ratios | |
|---|---|---|---|---|
| | % frequency | % frequency | N/T | T/N |
| Bladder | 0.0000 | 0.0000 | undef | undef |
| Breast | 0.0000 | 0.0000 | undef | undef |
| Small intestine | 0.0000 | 0.0000 | undef | undef |
| Ovary | 0.0000 | 0.0000 | undef | undef |
| Endocrine tissue | 0.0000 | 0.0050 | 0.0000 | undef |
| Gastrointestinal | 0.0000 | 0.0000 | undef | undef |
| Brain | 0.0007 | 0.0000 | undef | 0.0000 |
| Hematopoietic | 0.0000 | 0.0000 | undef | undef |
| Skin | 0.0000 | 0.0000 | undef | undef |
| Hepatic | 0.0000 | 0.0000 | undef | undef |
| Heart | 0.0000 | 0.0000 | undef | undef |
| Testicles | 0.0000 | 0.0000 | undef | undef |
| Lung | 0.0000 | 0.0000 | undef | undef |
| Stomach-esophagus | 0.0000 | 0.0000 | undef | undef |
| Muscle-skeleton | 0.0000 | 0.0000 | undef | undef |
| Kidney | 0.0000 | 0.0000 | undef | undef |
| Pancreas | 0.0248 | 0.0000 | undef | 0.0000 |
| Penis | 0.0000 | 0.0000 | undef | undef |
| Prostate | 0.0000 | 0.0000 | undef | undef |
| Uterus-endometrium | 0.0000 | 0.0000 | undef | undef |
| Uterus-myometrium | 0.0000 | 0.0000 | undef | undef |
| Uterus-general | 0.0000 | 0.0000 | undef | undef |
| Breast hyperplasia | 0.0000 | | | |
| Prostate hyperplasia | 0.0030 | | | |
| Seminal vesicle | 0.0000 | | | |
| Sensory organs | 0.0000 | | | |
| White blood cells | 0.0000 | | | |
| Cervix | 0.0000 | | | |

| | | STANDARDIZED/SUBTRACTED | |
|---|---|---|---|
| | FETUS | LIBRARIES | |
| | % frequency | % frequency | |
| Development | 0.0000 | Breast | 0.0000 |
| Gastrointestinal | 0.0056 | Ovary_n | 0.0000 |
| Brain | 0.0000 | Ovary_t | 0.0000 |
| Hematopoietic | 0.0000 | Endocrine tissue | 0.0000 |
| Skin | 0.0000 | Fetal | 0.0000 |
| Hepatic | 0.0000 | Gastrointestinal | 0.0122 |
| Heart-blood vessels | 0.0000 | Hematopoietic | 0.0000 |
| Lung | 0.0000 | Skin-muscle | 0.0000 |
| Suprarenal gland | 0.0000 | Testicles | 0.0000 |
| Kidney | 0.0000 | Lung | 0.0000 |
| Placenta | 0.0000 | Nerves | 0.0010 |
| Prostate | 0.0000 | Prostate | 0.0000 |
| Sensory organs | 0.0000 | Sensory Organs | 0.0000 |
| | | Uterus_n | 0.0000 |

Electronic Northern for SEQ. ID NO.: 25

| | NORMAL | TUMOR | Ratios | |
|---|---|---|---|---|
| | % frequency | % frequency | N/T | T/N |
| Bladder | 0.0000 | 0.0000 | undef | undef |
| Breast | 0.0000 | 0.0019 | 0.0000 | undef |
| Small intestine | 0.0000 | 0.0000 | undef | undef |
| Ovary | 0.0000 | 0.0000 | undef | undef |
| Endocrine tissue | 0.0000 | 0.0000 | undef | undef |
| Gastrointestinal | 0.0000 | 0.0000 | undef | undef |
| Brain | 0.0000 | 0.0000 | undef | undef |
| Hematopoietic | 0.0000 | 0.0000 | undef | undef |
| Skin | 0.0000 | 0.0000 | undef | undef |
| Hepatic | 0.0000 | 0.0000 | undef | undef |
| Heart | 0.0000 | 0.0000 | undef | undef |
| Testicles | 0.0000 | 0.0000 | undef | undef |
| Lung | 0.0000 | 0.0000 | undef | undef |
| Stomach-esophagus | 0.0000 | 0.0000 | undef | undef |
| Muscle-skeleton | 0.0000 | 0.0000 | undef | undef |
| Kidney | 0.0000 | 0.0000 | undef | undef |
| Pancreas | 0.2263 | 0.0055 | 40.9832 | 0.0244 |
| Penis | 0.0000 | 0.0000 | undef | undef |
| Prostate | 0.0000 | 0.0000 | undef | undef |
| Uterus-endometrium | 0.0000 | 0.0000 | undef | undef |
| Uterus-myometrium | 0.0000 | 0.0000 | undef | undef |
| Uterus-general | 0.0000 | 0.0000 | undef | undef |
| Breast hyperplasia | 0.0000 | | | |
| Prostate hyperplasia | 0.0000 | | | |
| Seminal vesicle | 0.0000 | | | |
| Sensory organs | 0.0000 | | | |
| White blood cells | 0.0000 | | | |
| Cervix | 0.0000 | | | |

| | | STANDARDIZED/SUBTRACTED | |
|---|---|---|---|
| | FETUS | LIBRARIES | |
| | % frequency | % frequency | |
| Development | 0.0000 | Breast | 0.0000 |
| Gastrointestinal | 0.0528 | Ovary_n | 0.0000 |
| Brain | 0.0000 | Ovary_t | 0.0000 |
| Hematopoietic | 0.0000 | Endocrine tissue | 0.0000 |
| Skin | 0.0000 | Fetal | 0.0000 |
| Hepatic | 0.0000 | Gastrointestinal | 0.0000 |
| Heart-blood vessels | 0.0000 | Hematopoietic | 0.0000 |
| Lung | 0.0000 | Skin-muscle | 0.0000 |
| Suprarenal gland | 0.0000 | Testicles | 0.0000 |
| Kidney | 0.0000 | Lung | 0.0000 |
| Placenta | 0.0000 | Nerves | 0.0000 |
| Prostate | 0.0000 | Prostate | 0.0000 |
| Sensory organs | 0.0000 | Sensory Organs | 0.0000 |
| | | Uterus_n | 0.0000 |

Electronic Northern for SEQ. ID NO.: 27

| | NORMAL | TUMOR | Ratios | |
|---|---|---|---|---|
| | % frequency | % frequency | N/T | T/N |
| Bladder | 0.0000 | 0.0000 | undef | undef |
| Breast | 0.0000 | 0.0000 | undef | undef |
| Small intestine | 0.0000 | 0.0000 | undef | undef |
| Ovary | 0.0000 | 0.0000 | undef | undef |
| Endocrine tissue | 0.0000 | 0.0000 | undef | undef |
| Gastrointestinal | 0.0000 | 0.0000 | undef | undef |
| Brain | 0.0000 | 0.0000 | undef | undef |
| Hematopoietic | 0.0000 | 0.0000 | undef | undef |
| Skin | 0.0000 | 0.0000 | undef | undef |
| Hepatic | 0.0000 | 0.0000 | undef | undef |
| Heart | 0.0000 | 0.0000 | undef | undef |
| Testicles | 0.0000 | 0.0000 | undef | undef |
| Lung | 0.0000 | 0.0000 | undef | undef |
| Stomach-esophagus | 0.0000 | 0.0000 | undef | undef |
| Muscle-skeleton | 0.0000 | 0.0000 | undef | undef |
| Kidney | 0.0380 | 0.0055 | 6.8804 | 0.1453 |
| Pancreas | 0.0000 | 0.0000 | undef | undef |
| Penis | 0.0000 | 0.0000 | undef | undef |
| Prostate | 0.0000 | 0.0000 | undef | undef |
| Uterus-endometrium | 0.0000 | 0.0000 | undef | undef |
| Uterus-myometrium | 0.0000 | 0.0000 | undef | undef |
| Uterus-general | 0.0000 | 0.0000 | undef | undef |
| Breast hyperplasia | 0.0000 | | | |
| Prostate hyperplasia | 0.0000 | | | |
| Seminal vesicle | 0.0000 | | | |
| Sensory organs | 0.0000 | | | |
| White blood cells | 0.0000 | | | |
| Cervix | 0.0000 | | | |

| | | STANDARDIZED/SUBTRACTED | |
|---|---|---|---|
| | FETUS | LIBRARIES | |
| | % frequency | % frequency | |
| Development | 0.0000 | Breast | 0.0000 |
| Gastrointestinal | 0.0000 | Ovary_n | 0.0000 |
| Brain | 0.0000 | Ovary_t | 0.0000 |
| Hematopoietic | 0.0000 | Endocrine tissue | 0.0000 |
| Skin | 0.0000 | Fetal | 0.0000 |
| Hepatic | 0.0000 | Gastrointestinal | 0.0000 |
| Heart-blood vessels | 0.0000 | Hematopoietic | 0.0000 |
| Lung | 0.0000 | Skin-muscle | 0.0000 |
| Suprarenal gland | 0.0000 | Testicles | 0.0000 |
| Kidney | 0.0000 | Lung | 0.0000 |
| Placenta | 0.0000 | Nerves | 0.0000 |
| Prostate | 0.0000 | Prostate | 0.0000 |
| Sensory organs | 0.0000 | Sensory Organs | 0.0000 |
| | | Uterus_n | 0.0000 |

Electronic Northern for SEQ. ID NO.: 28

| | NORMAL | TUMOR | Ratios | |
|---|---|---|---|---|
| | % frequency | % frequency | N/T | T/N |
| Bladder | 0.0000 | 0.0000 | undef | undef |
| Breast | 0.0000 | 0.0000 | undef | undef |
| Small intestine | 0.0000 | 0.0000 | undef | undef |
| Ovary | 0.0000 | 0.0000 | undef | undef |
| Endocrine tissue | 0.0000 | 0.0000 | undef | undef |
| Gastrointestinal | 0.0000 | 0.0000 | undef | undef |
| Brain | 0.0000 | 0.0000 | undef | undef |
| Hematopoietic | 0.0000 | 0.0000 | undef | undef |
| Skin | 0.0000 | 0.0000 | undef | undef |
| Hepatic | 0.0000 | 0.0000 | undef | undef |
| Heart | 0.0000 | 0.0000 | undef | undef |
| Testicles | 0.0000 | 0.0000 | undef | undef |
| Lung | 0.0000 | 0.0000 | undef | undef |
| Stomach-esophagus | 0.0000 | 0.0000 | undef | undef |
| Muscle-skeleton | 0.0000 | 0.0000 | undef | undef |
| Kidney | 0.0446 | 0.0000 | undef | 0.0000 |
| Pancreas | 0.0000 | 0.0000 | undef | undef |
| Penis | 0.0000 | 0.0000 | undef | undef |
| Prostate | 0.0000 | 0.0000 | undef | undef |
| Uterus-endometrium | 0.0000 | 0.0000 | undef | undef |
| Uterus-myometrium | 0.0000 | 0.0000 | undef | undef |
| Uterus-general | 0.0000 | 0.0000 | undef | undef |
| Breast hyperplasia | 0.0000 | | | |
| Prostate hyperplasia | 0.0000 | | | |
| Seminal vesicle | 0.0000 | | | |
| Sensory organs | 0.0000 | | | |
| White blood cells | 0.0000 | | | |
| Cervix | 0.0000 | | | |

| | | STANDARDIZED/SUBTRACTED | |
|---|---|---|---|
| | FETUS | LIBRARIES | |
| | % frequency | % frequency | |
| Development | 0.0000 | Breast | 0.0000 |
| Gastrointestinal | 0.0056 | Ovary_n | 0.0000 |
| Brain | 0.0000 | Ovary_t | 0.0000 |
| Hematopoietic | 0.0000 | Endocrine tissue | 0.0000 |
| Skin | 0.0000 | Fetal | 0.0000 |
| Hepatic | 0.0000 | Gastrointestinal | 0.0000 |
| Heart-blood vessels | 0.0000 | Hematopoietic | 0.0000 |
| Lung | 0.0000 | Skin-muscle | 0.0000 |
| Suprarenal gland | 0.0000 | Testicles | 0.0000 |
| Kidney | 0.0000 | Lung | 0.0000 |
| Placenta | 0.0000 | Nerves | 0.0000 |
| Prostate | 0.0000 | Prostate | 0.0000 |
| Sensory organs | 0.0000 | Sensory Organs | 0.0000 |
| | | Uterus_n | 0.0000 |

Electronic Northern for SEQ. ID NO.: 29

| | NORMAL | TUMOR | Ratios | |
|---|---|---|---|---|
| | % frequency | % frequency | N/T | T/N |
| Bladder | 0.0000 | 0.0000 | undef | undef |
| Breast | 0.0000 | 0.0000 | undef | undef |
| Small intestine | 0.0000 | 0.0000 | undef | undef |
| Ovary | 0.0000 | 0.0000 | undef | undef |
| Endocrine tissue | 0.0000 | 0.0000 | undef | undef |
| Gastrointestinal | 0.0000 | 0.0000 | undef | undef |
| Brain | 0.0000 | 0.0000 | undef | undef |
| Hematopoietic | 0.0000 | 0.0000 | undef | undef |
| Skin | 0.0000 | 0.0000 | undef | undef |
| Hepatic | 0.0000 | 0.0000 | undef | undef |
| Heart | 0.0000 | 0.0000 | undef | undef |
| Testicles | 0.0000 | 0.0000 | undef | undef |
| Lung | 0.0000 | 0.0000 | undef | undef |
| Stomach-esophagus | 0.0000 | 0.0000 | undef | undef |
| Muscle-skeleton | 0.0000 | 0.0000 | undef | undef |
| Kidney | 0.0248 | 0.0000 | undef | 0.0000 |
| Pancreas | 0.0000 | 0.0000 | undef | undef |
| Penis | 0.0000 | 0.0000 | undef | undef |
| Prostate | 0.0000 | 0.0000 | undef | undef |
| Uterus-endometrium | 0.0000 | 0.0000 | undef | undef |
| Uterus-myometrium | 0.0000 | 0.0000 | undef | undef |
| Uterus-general | 0.0000 | 0.0000 | undef | undef |
| Breast hyperplasia | 0.0000 | | | |
| Prostate hyperplasia | 0.0000 | | | |
| Seminal vesicle | 0.0000 | | | |
| Sensory organs | 0.0000 | | | |
| White blood cells | 0.0000 | | | |
| Cervix | 0.0000 | | | |

| | | STANDARDIZED/SUBTRACTED | |
|---|---|---|---|
| | FETUS | LIBRARIES | |
| | % frequency | % frequency | |
| Development | 0.0000 | Breast | 0.0000 |
| Gastrointestinal | 0.0000 | Ovary_n | 0.0000 |
| Brain | 0.0000 | Ovary_t | 0.0000 |
| Hematopoietic | 0.0000 | Endocrine tissue | 0.0000 |
| Skin | 0.0000 | Fetal | 0.0000 |
| Hepatic | 0.0000 | Gastrointestinal | 0.1220 |
| Heart-blood vessels | 0.0000 | Hematopoietic | 0.0000 |
| Lung | 0.0000 | Skin-muscle | 0.0000 |
| Suprarenal gland | 0.0000 | Testicles | 0.0000 |
| Kidney | 0.0000 | Lung | 0.0000 |
| Placenta | 0.0000 | Nerves | 0.0000 |
| Prostate | 0.0000 | Prostate | 0.0000 |
| Sensory organs | 0.0000 | Sensory Organs | 0.0000 |
| | | Uterus_n | 0.0000 |

Electronic Northern for SEQ. ID NO.: 30

| | NORMAL | TUMOR | Ratios | |
|---|---|---|---|---|
| | % frequency | % frequency | N/T | T/N |
| Bladder | 0.0000 | 0.0026 | 0.0000 | undef |
| Breast | 0.0000 | 0.0000 | undef | undef |
| Small intestine | 0.0000 | 0.0000 | undef | undef |
| Ovary | 0.0000 | 0.0000 | undef | undef |
| Endocrine tissue | 0.0000 | 0.0000 | undef | undef |
| Gastrointestinal | 0.0000 | 0.0000 | undef | undef |
| Brain | 0.0000 | 0.0000 | undef | 0.0000 |
| Hematopoietic | 0.0000 | 0.0000 | undef | undef |
| Skin | 0.0000 | 0.0000 | undef | undef |
| Hepatic | 0.0000 | 0.0000 | undef | undef |
| Heart | 0.0000 | 0.0000 | undef | undef |
| Testicles | 0.0000 | 0.0000 | undef | undef |
| Lung | 0.0000 | 0.0000 | undef | undef |
| Stomach-esophagus | 0.0000 | 0.0000 | undef | undef |
| Muscle-skeleton | 0.0000 | 0.0000 | undef | undef |
| Kidney | 0.2693 | 0.0110 | 24.3805 | 0.0410 |
| Pancreas | 0.0000 | 0.0000 | undef | undef |
| Penis | 0.0022 | 0.0000 | undef | 0.0000 |
| Prostate | 0.0000 | 0.0000 | undef | undef |
| Uterus-endometrium | 0.0000 | 0.0000 | undef | undef |
| Uterus-myometrium | 0.0000 | 0.0000 | undef | undef |
| Uterus-general | 0.0000 | 0.0000 | undef | undef |
| Breast hyperplasia | 0.0000 | | | |
| Prostate hyperplasia | 0.0000 | | | |
| Seminal vesicle | 0.0000 | | | |
| Sensory organs | 0.0000 | | | |
| White blood cells | 0.0000 | | | |
| Cervix | 0.0000 | | | |

| | | STANDARDIZED/SUBTRACTED | |
|---|---|---|---|
| | FETUS | LIBRARIES | |
| | % frequency | % frequency | |
| Development | 0.0000 | Breast | 0.0000 |
| Gastrointestinal | 0.0028 | Ovary_n | 0.0000 |
| Brain | 0.0000 | Ovary_t | 0.0000 |
| Hematopoietic | 0.0000 | Endocrine tissue | 0.0000 |
| Skin | 0.0000 | Fetal | 0.0000 |
| Hepatic | 0.0000 | Gastrointestinal | 0.0000 |
| Heart-blood vessels | 0.0000 | Hematopoietic | 0.0000 |
| Lung | 0.0000 | Skin-muscle | 0.0000 |
| Suprarenal gland | 0.0000 | Testicles | 0.0000 |
| Kidney | 0.0000 | Lung | 0.0000 |
| Placenta | 0.0000 | Nerves | 0.0010 |
| Prostate | 0.0000 | Prostate | 0.0000 |
| Sensory organs | 0.0000 | Sensory Organs | 0.0000 |
| | | Uterus_n | 0.0000 |

Electronic Northern for SEQ. ID NO.: 31

| | NORMAL | TUMOR | Ratios | |
|---|---|---|---|---|
| | % frequency | % frequency | N/T | T/N |
| Bladder | 0.0000 | 0.0000 | undef | undef |
| Breast | 0.0000 | 0.0000 | undef | undef |
| Small intestine | 0.0000 | 0.0000 | undef | undef |
| Ovary | 0.0000 | 0.0000 | undef | undef |
| Endocrine tissue | 0.0000 | 0.0000 | undef | undef |
| Gastrointestinal | 0.0000 | 0.0000 | undef | undef |
| Brain | 0.0000 | 0.0000 | undef | undef |
| Hematopoietic | 0.0013 | 0.0000 | undef | 0.0000 |
| Skin | 0.0000 | 0.0000 | undef | undef |
| Hepatic | 0.0000 | 0.0000 | undef | undef |
| Heart | 0.0000 | 0.0000 | undef | undef |
| Testicles | 0.0000 | 0.0000 | undef | undef |
| Lung | 0.0000 | 0.0000 | undef | undef |
| Stomach-esophagus | 0.0000 | 0.0000 | undef | undef |
| Muscle-skeleton | 0.0000 | 0.0000 | undef | undef |
| Kidney | 0.0000 | 0.0000 | undef | undef |
| Pancreas | 0.1850 | 0.0110 | 16.7523 | 0.0597 |
| Penis | 0.0000 | 0.0000 | undef | undef |
| Prostate | 0.0000 | 0.0000 | undef | undef |
| Uterus-endometrium | 0.0000 | 0.0000 | undef | undef |
| Uterus-myometrium | 0.0000 | 0.0000 | undef | undef |
| Uterus-general | 0.0000 | | | |
| Breast hyperplasia | 0.0000 | | | |
| Prostate hyperplasia | 0.0000 | | | |
| Seminal vesicle | 0.0000 | | | |
| Sensory organs | 0.0000 | | | |
| White blood cells | 0.0000 | | | |
| Cervix | 0.0000 | | | |

| | | STANDARDIZED/SUBTRACTED | |
|---|---|---|---|
| | FETUS | LIBRARIES | |
| | % frequency | % frequency | |
| Development | 0.0000 | Breast | 0.0000 |
| Gastrointestinal | 0.0000 | Ovary_n | 0.0000 |
| Brain | 0.0000 | Ovary_t | 0.0000 |
| Hematopoietic | 0.0000 | Endocrine tissue | 0.0000 |
| Skin | 0.0000 | Fetal | 0.0000 |
| Hepatic | 0.0000 | Gastrointestinal | 0.0000 |
| Heart-blood vessels | 0.0000 | Hematopoietic | 0.0000 |
| Lung | 0.0000 | Skin-muscle | 0.0000 |
| Suprarenal gland | 0.0000 | Testicles | 0.0000 |
| Kidney | 0.0000 | Lung | 0.0000 |
| Placenta | 0.0061 | Nerves | 0.0000 |
| Prostate | 0.0000 | Prostate | 0.0000 |
| Sensory organs | 0.0000 | Sensory Organs | 0.0000 |
| | | Uterus_n | 0.0000 |

Electronic Northern for SEQ. ID NO.: 35

| | NORMAL | TUMOR | Ratios | |
|---|---|---|---|---|
| | % frequency | % frequency | N/T | T/N |
| Bladder | 0.0000 | 0.0000 | undef | undef |
| Breast | 0.0000 | 0.0000 | undef | undef |
| Small intestine | 0.0000 | 0.0000 | undef | undef |
| Ovary | 0.0000 | 0.0000 | undef | undef |
| Endocrine tissue | 0.0000 | 0.0000 | undef | undef |
| Gastrointestinal | 0.0000 | 0.0000 | undef | undef |
| Brain | 0.0000 | 0.0000 | undef | undef |
| Hematopoietic | 0.0000 | 0.0000 | undef | undef |
| Skin | 0.0000 | 0.0000 | undef | undef |
| Hepatic | 0.0000 | 0.0000 | undef | undef |
| Heart | 0.0000 | 0.0000 | undef | undef |
| Testicles | 0.0000 | 0.0000 | undef | undef |
| Lung | 0.0000 | 0.0000 | undef | undef |
| Stomach-esophagus | 0.0000 | 0.0000 | undef | undef |
| Muscle-skeleton | 0.0000 | 0.0000 | undef | undef |
| Kidney | 0.0000 | 0.0000 | undef | undef |
| Pancreas | 0.0380 | 0.0000 | undef | 0.0000 |
| Penis | 0.0000 | 0.0000 | undef | undef |
| Prostate | 0.0000 | 0.0000 | undef | undef |
| Uterus-endometrium | 0.0000 | 0.0000 | undef | undef |
| Uterus-myometrium | 0.0000 | 0.0000 | undef | undef |
| Uterus-general | 0.0000 | 0.0000 | undef | undef |
| Breast hyperplasia | 0.0000 | | | |
| Prostate hyperplasia | 0.0000 | | | |
| Seminal vesicle | 0.0000 | | | |
| Sensory organs | 0.0000 | | | |
| White blood cells | 0.0000 | | | |
| Cervix | 0.0000 | | | |

| | | STANDARDIZED/SUBTRACTED | |
|---|---|---|---|
| | FETUS | LIBRARIES | |
| | % frequency | % frequency | |
| Development | 00000 | Breast | 0.0000 |
| Gastrointestinal | 0.0083 | Ovary_n | 0.0000 |
| Brain | 0.0000 | Ovary_t | 0.0000 |
| Hematopoietic | 0.0000 | Endocrine tissue | 0.0000 |
| Skin | 0.0000 | Fetal | 0.0000 |
| Hepatic | 0.0000 | Gastrointestinal | 0.0000 |
| Heart-blood vessels | 0.0000 | Hematopoietic | 0.0000 |
| Lung | 0.0000 | Skin-muscle | 0.0000 |
| Suprarenal gland | 0.0000 | Testicles | 0.0000 |
| Kidney | 0.0000 | Lung | 0.0000 |
| Placenta | 0.0000 | Nerves | 0.0000 |
| Prostate | 0.0000 | Prostate | 0.0000 |
| Sensory organs | 0.0000 | Sensory Organs | 0.0000 |
| | | Uterus_n | 0.0000 |

Electronic Northern for SEQ. ID NO.: 36

| | NORMAL | TUMOR | Ratios | |
|---|---|---|---|---|
| | % frequency | % frequency | N/T | T/N |
| Bladder | 0.0000 | 0.0026 | 0.0000 | undef |
| Breast | 0.0000 | 0.0000 | undef | undef |
| Small intestine | 0.0000 | 0.0000 | undef | undef |
| Ovary | 0.0000 | 0.0000 | undef | undef |
| Endocrine tissue | 0.0000 | 0.0000 | undef | undef |
| Gastrointestinal | 0.0000 | 0.0000 | undef | undef |
| Brain | 0.0000 | 0.0000 | undef | undef |
| Hematopoietic | 0.0000 | 0.0000 | undef | undef |
| Skin | 0.0000 | 0.0000 | undef | undef |
| Hepatic | 0.0000 | 0.0000 | undef | undef |
| Heart | 0.0000 | 0.0000 | undef | undef |
| Testicles | 0.0000 | 0.0000 | undef | undef |
| Lung | 0.0000 | 0.0000 | undef | undef |
| Stomach-esophagus | 0.0000 | 0.0000 | undef | undef |
| Muscle-skeleton | 0.0000 | 0.0000 | undef | undef |
| Kidney | 0.0000 | 0.0000 | undef | undef |
| Pancreas | 0.3354 | 0.0166 | 20.2423 | 0.0494 |
| Penis | 0.0000 | 0.0000 | undef | undef |
| Prostate | 0.0022 | 0.0000 | undef | 0.0000 |
| Uterus-endometrium | 0.0000 | 0.0000 | undef | undef |
| Uterus-myometrium | 0.0000 | 0.0000 | undef | undef |
| Uterus-general | 0.0000 | 0.0000 | undef | undef |
| Breast hyperplasia | 0.0000 | | | |
| Prostate hyperplasia | 0.0000 | | | |
| Seminal vesicle | 0.0000 | | | |
| Sensory organs | 0.0000 | | | |
| White blood cells | 0.0000 | | | |
| Cervix | 0.0000 | | | |

| | | STANDARDIZED/SUBTRACTED | |
|---|---|---|---|
| | FETUS | LIBRARIES | |
| | % frequency | % frequency | |
| Development | 0.0000 | Breast | 0.0000 |
| Gastrointestinal | 0.0028 | Ovary_n | 0.0000 |
| Brain | 0.0000 | Ovary_t | 0.0000 |
| Hematopoietic | 0.0000 | Endocrine tissue | 0.0000 |
| Skin | 0.0000 | Fetal | 0.0000 |
| Hepatic | 0.0000 | Gastrointestinal | 0.0000 |
| Heart-blood vessels | 0.0000 | Hematopoietic | 0.0000 |
| Lung | 0.0000 | Skin-muscle | 0.0000 |
| Suprarenal gland | 0.0000 | Testicles | 0.0000 |
| Kidney | 0.0000 | Lung | 0.0000 |
| Placenta | 0.0000 | Nerves | 0.0000 |
| Prostate | 0.0000 | Prostate | 0.0000 |
| Sensory organs | 0.0000 | Sensory Organs | 0.0000 |
| | | Uterus_n | 0.0000 |

Electronic Northern for SEQ. ID NO.: 37

| | NORMAL | TUMOR | Ratios | |
|---|---|---|---|---|
| | % frequency | % frequency | N/T | T/N |
| Bladder | 0.0000 | 0.0000 | undef | undef |
| Breast | 0.0000 | 0.0000 | undef | undef |
| Small intestine | 0.0000 | 0.0000 | undef | undef |
| Ovary | 0.0000 | 0.0000 | undef | undef |
| Endocrine tissue | 0.0000 | 0.0000 | undef | undef |
| Gastrointestinal | 0.0000 | 0.0000 | undef | undef |
| Brain | 0.0000 | 0.0000 | undef | undef |
| Hematopoietic | 0.0000 | 0.0000 | undef | undef |
| Skin | 0.0000 | 0.0000 | undef | undef |
| Hepatic | 0.0000 | 0.0000 | undef | undef |
| Heart | 0.0000 | 0.0000 | undef | undef |
| Testicles | 0.0000 | 0.0000 | undef | undef |
| Lung | 0.0000 | 0.0000 | undef | undef |
| Stomach-esophagus | 0.0000 | 0.0000 | undef | undef |
| Muscle-skeleton | 0.0000 | 0.0000 | undef | undef |
| Kidney | 0.0000 | 0.0000 | undef | undef |
| Pancreas | 0.0198 | 0.0000 | undef | 0.0000 |
| Penis | 0.0000 | 0.0000 | undef | undef |
| Prostate | 0.0000 | 0.0000 | undef | undef |
| Uterus-endometrium | 0.0000 | 0.0000 | undef | undef |
| Uterus-myometrium | 0.0000 | 0.0000 | undef | undef |
| Uterus-general | 0.0000 | 0.0000 | undef | undef |
| Breast hyperplasia | 0.0000 | | | |
| Prostate hyperplasia | 0.0000 | | | |
| Seminal vesicle | 0.0000 | | | |
| Sensory organs | 0.0000 | | | |
| White blood cells | 0.0000 | | | |
| Cervix | 0.0000 | | | |

| | | STANDARDIZED/SUBTRACTED | |
|---|---|---|---|
| | FETUS | LIBRARIES | |
| | % frequency | % frequency | |
| Development | 0.0000 | Breast | 0.0000 |
| Gastrointestinal | 0.0000 | Ovary_n | 0.0000 |
| Brain | 0.0000 | Ovary_t | 0.0000 |
| Hematopoietic | 0.0000 | Endocrine tissue | 0.0000 |
| Skin | 0.0000 | Fetal | 0.0000 |
| Hepatic | 0.0000 | Gastrointestinal | 0.0000 |
| Heart-blood vessels | 0.0000 | Hematopoietic | 0.0000 |
| Lung | 0.0000 | Skin-muscle | 0.0000 |
| Suprarenal gland | 0.0000 | Testicles | 0.0000 |
| Kidney | 0.0000 | Lung | 0.0000 |
| Placenta | 0.0000 | Nerves | 0.0000 |
| Prostate | 0.0000 | Prostate | 0.0000 |
| Sensory organs | 0.0000 | Sensory Organs | 0.0000 |
| | | Uterus_n | 0.0000 |

Electronic Northern for Seq. ID: 67

| | NORMAL | TUMOR | Ratios | |
|---|---|---|---|---|
| | % frequency | % frequency | N/T | T/N |
| B lymphoma | 0.0000 | 0.0000 | undef | undef |
| Bladder | 0.0000 | 0.0000 | undef | undef |
| Breast | 0.0000 | 0.0000 | undef | undef |
| Large intestine | 0.0000 | 0.0000 | undef | undef |
| Small intestine | 0.0000 | 0.0000 | undef | undef |
| Ovary | 0.0000 | 0.0000 | undef | undef |
| Endocrine tissue | 0.0000 | 0.0000 | undef | undef |
| Brain | 0.0000 | 0.0000 | undef | undef |
| Skin | 0.0000 | 0.0000 | undef | undef |
| Hepatic | 0.0000 | 0.0000 | undef | undef |
| Heart | 0.0000 | 0.0000 | undef | undef |
| Testicles | 0.0000 | 0.0000 | undef | undef |
| Lung | 0.0000 | 0.0000 | undef | undef |
| Stomach-esophagus | 0.0000 | 0.0000 | undef | undef |
| Muscle-skeleton | 0.0000 | 0.0000 | undef | undef |
| Kidney | 0.0000 | 0.0000 | undef | undef |
| Pancreas | 0.1999 | 0.0055 | 36.1978 | 0.0276 |
| Prostate | 0.0000 | 0.0013 | 0.0000 | undef |
| T lymphoma | 0.0051 | 0.0000 | undef | 0.0000 |
| Uterus | 0.0000 | 0.0000 | undef | undef |
| White blood cells | 0.0014 | 0.0000 | undef | 0.0000 |
| Hematopoietic | 0.0000 | | | |
| Penis | 0.0000 | | | |
| Seminal vescicle | 0.0000 | | | |
| Sensory organs | 0.0000 | | | |

| | FETUS |
|---|---|
| | % freq. |
| Development | 0.0000 |
| Gastrointestinal | 0.0000 |
| Brain | 0.0000 |
| Hematopoietic | 0.0000 |
| Skin | 0.0000 |
| Hepatic | 0.0000 |
| Heart-blood vessels | 0.0000 |
| Lung | 0.0000 |
| Adrenal gland | 0.0000 |
| Kidney | 0.0000 |
| Placenta | 0.0000 |
| Prostate | 0.0000 |
| Sensory organs | 0.0000 |
| | STANDARDIZED/SUBTRACTED |
| | LIBRARIES % frequency |
| Breast | 0.0000 |
| Breast_t | 0.0000 |
| Large Intestine_t | 0.0000 |
| Ovary_n | 0.0000 |
| Ovary_t | 0.0000 |
| Endocrine tissue | 0.0000 |
| Fetal | 0.0000 |
| Gastrointestinal | 0.0244 |
| Hematopoietic | 0.0000 |
| Skin-muscle | 0.0000 |
| Testicles_n | 0.0000 |
| Testicles_t | 0.0000 |
| Lungs_n | 0.0000 |
| Lungs_t | 0.0000 |
| Nerves | 0.0000 |
| Kidney_t | 0.0000 |
| Ovary_uterus | 0.0000 |
| Prostate_n | 0.0000 |
| Sensory organs | 0.0000 |
| White blood cells | 0.0000 |

2.2. Fisher Test

In order to decide whether a partial sequence S of a gene occurs significantly more often or less often in a library for normal tissue than in a library for degenerated tissue, Fisher's exact test, a standard statistical process, is carried out (Hays, W. L., (1991) Statistics, Harcourt Brace College Publishers, Fort Worth).

The null hypothesis reads: The two libraries cannot be distinguished with respect to the frequency of sequences homologous to S. If the null hypothesis can be rejected with high enough certainty, the gene belonging to S is accepted as an advantageous candidate for a cancer gene, and in the next step an attempt is made to achieve lengthening of its sequence.

### EXAMPLE 3

Automatic Lengthening of the Partial Sequence

Automatic lengthening of partial sequence S is completed in three steps:

1. Determination of all sequences homologous to S from the total set of available sequences using BLAST

2. Assembling these sequences by means of the standard program GAP4 (Bonfield, J. K.; Smith, K. F. and Staden, R. (1995), Nucleic Acids Research 23 4992-4999) (contig formation).

3. Computation of a consensus sequence C from the assembled sequences.

Consensus sequence C will generally be longer than initial sequence S. Its electronic Northern Blot will accordingly deviate from that for S. A repeated Fisher test decides whether the alternative hypothesis of deviation from a uniform expression in the two libraries can be maintained. If this is the case, an attempt is made to lengthen C in the same way as S. This iteration is continued with consensus sequences Cᵢ (i: iteration index) obtained in each case until the alternative hypothesis is rejected (if H₀ Exit; truncation criterion I) or until automatic lengthening is no longer possible (while Cᵢ>Cᵢ₋₁; truncation criterion II)

In the case of truncation criterion II, with the consensus sequence present after the last iteration, a complete or roughly complete sequence of a gene which can be related to cancer with high statistical certainty is acquired.

Analogously to the above-described examples, it was possible to find from normal pancreatic tissue the nucleic acid sequences described in Table I.

Furthermore, for the individual nucleic acid sequences, it was possible to determine the peptide sequences (ORFs) that are listed in Table II, in which no peptide can be assigned to a few nucleic acid sequences and more than one peptide can be assigned to some nucleic acid sequences. As already mentioned above, both the determined nucleic acid sequences and the peptide sequences assigned to the nucleic acid sequences are the subject of this invention.

### EXAMPLE 4

Mapping of Nucleic Acid Sequences on the Human Genome

Human genes were mapped using the Stanford G3 Hybrid Panel (Stewart et al., 1997), which is marketed by Research Genetics, Huntsville, Ala. This panel consists of 83 different genomic DNAs of human-hamster hybrid cell lines and allows resolution of 500 kilobases. The hybrid cell lines were obtained by fusion of irradiated diploid human cells with cells of the Chinese hamster. The retention pattern of the human chromosome fragments is determined by means of gene-specific primers in a polymerase chain reaction and is analyzed using software available from the Stanford RH server (http://www.stanford.edu/RH/rhserver_form2.html). This program determines the STS marker that is nearest to the desired gene. The corresponding cytogenetic band was determined using the “Mapview” program of the Genome Database (GDB), (http://gdbwww.dkfz-heidelberg.de).

In addition to mapping of genes on the human chromosome set by various experimental methods, it is possible to determine the location of genes on this by biocomputer methods. To do this, the known program e-PCR was used (Schuler GD (1998) Electronic PCR: Bridging the gap between genome mapping and genome sequencing. Trends Biotechnol 16: 456-459, Schuler GD (1997). Sequence mapping by electronic PCR. Genome Res. 7: 541-550). The database used here no longer corresponds to the one cited in the literature, but is a further development which includes data from the public database RHdb (http://www.ebi.ac.uk/RHdb/-index.html). Analogously to the mapping by the hybrid panels, the results were evaluated with the above-mentioned software and the software of the Whitehead-Institute (http://carbon.wi.mit.edu:8000/cgi-bin/contig/rhmapper.p1).

### EXAMPLE 5

Obtaining Genomic DNA Sequences (BAC Clones)

The genomic BAC clones that contain the corresponding cDNAs (http://www.tree.caltech.edu/; Shizuya, H.; B. Birren, U -J. Kim, V. Mancino, T. Slepak, Y. Tachiiri, M. Simon (1992) Proc. Natl. Acad. Sci., USA 89: 8794-8797) were isolated with the procedure of “down-to-the-well”. In this procedure, a library consisting of BAC clones (the library covers roughly 3× the human genome) is moved into a certain raster, so that the DNA of these clones with a specific PCR can be studied. In doing so, “pooling” of the DNA of different BAC clones takes place. Combinatorial analysis makes it possible to determine the clones that contain the desired DNA. By fixing the clones, the address of the clones in the library can be determined. This address together with the name of the library which is being used unequivocally fixes the clones and thus the DNA sequence of these clones.

TABLE I

| | |
|---|---|
| Col. 1 | Seq. ID No. |
| Col. 2 | Expresssion |
| Col. 3 | Function |
| Col. 4 | Modules |
| Col. 5 | Cytogenetic localization |
| Col. 6 | Nearest marker |

TABLE I

| Seq. | | |
|---|---|---|
| ID | | |
| No. | Expression | Function |
| 2 | Overexpressed in normal | Human mRNA for |
| | pancreatic tissue | regenerating protein I |
| | | beta |
| 4 | Overexpressed in normal | Glandular kallikrein 1 |
| | pancreatic tissue | |
| 6 | Overexpressed in normal | cDNA encoding human |
| | pancreatic tissue | phospholipase A2 |
| 7 | Overexpressed in normal | Human somatostatin I |
| | pancreatic tissue | |
| 9 | Overexpressed in normal | Human islet amyloid |
| | pancreatic tissue | protein |
| 11 | Overexpressed in normal | Human pancreatic secretory |
| | pancreatic tissue | trypsin inhibitor (PSTI) |
| 12 | Overexpressed in normal | Human protease E |
| | pancreatic tissue | |
| 14 | Overexpressed in normal | Homolog to trypsin |
| | pancreatic tissue | precursor |
| 15 | Overexpressed in normal | Human chymotrypsinogen |
| | pancreatic tissue | |
| 16 | Overexpressed in normal | Homo sapiens colipase |
| | pancreatic tissue | (CLPS) |
| 17 | Overexpressed in normal | caldecrin = serum calcium- |
| | pancreatic tissue | decreasing factor |
| 18 | Overexpressed in normal | Human pancreatic zymogen |
| | pancreatic tissue | granule membrane protein |
| | | GP-2 |
| 19 | Overexpressed in normal | Human IAPP |
| | pancreatic tissue | |
| 21 | Overexpressed in normal | H. sapiens mRNA for |
| | pancreatic tissue | chymotrypsin-like protease |
| | | CTRL-1 |
| 24 | Overexpressed in normal | Human homolog to D. |
| | pancreatic tissue | melanogaster Calbindin-32 |
| 25 | Overexpressed in normal | Human homolog to R. |
| | pancreatic tissue | norvegicus syncollin |
| 27 | Overexpressed in normal | Mus musculus unknown |
| | pancreatic tissue | protein precursor |
| 28 | Overexpressed in normal | Unknown |
| | pancreatic tissue | |
| 29 | Overexpressed in normal | Unknown |
| | pancreatic tissue | |
| 30 | Overexpressed in normal | Unknown |
| | pancreatic tissue | |
| 31 | Overexpressed in normal | Unknown |
| | pancreatic tissue | |
| 35 | Overexpressed in normal | Unknown |
| | pancreatic tissue | |
| 36 | Overexpressed in normal | Unknown |
| | pancreatic tissue | |
| 37 | Overexpressed in normal | Unknown |
| | pancreatic tissue | |
| 67 | Overexpressed in normal | Lengthening to Sequence |
| | pancreatic tissue | ID: 27 |

TABLE I

| | | | | Cytogenetische | |
|---|---|---|---|---|---|
| Seq ID No. | Expression | Funktion | Module | Lokalisation | Nächster Marker |
| 2 | in Pankreasnormalgewebe | Human mRNA for regenerating protein I beta | C_TYPE_{—} | 2p13.1 | CHLC.GCT1B4 |
| | überexprimiert | | LECTIN_2 | | |
| 4 | in Pankreasnormalgewebe | Glanduläres Kallikrein 1 | trypsin | | |
| | überexprimiert | | | | |
| 6 | in Pankreasnormalgewebe | cDNA encoding human phospholipase A2 | phoslip | 12q24.22-q24.23 | SHGC-10488- |
| | überexprimiert | | | | AFMa225xe5 |
| 7 | in Pankreasnormalgewebe | Human somalostatin I | | 3q26.33-q28 | D3S1262-D3S1580 |
| | überexprimiert | | | | |
| 9 | in Pankreasnormalgewebe | Human islet amyloid protein | Calc_CGRP_I | 12p12.3-p12.2 | D12S364-D12S310 |
| | überexprimiert | | APP | | |
| 11 | in Pankreasnormalgewebe | Human pancreatic secretory trypsin inhibitor | kazal | 12p12.3 | WI-7377-D12S1669 |
| | überexprimiert | (PSTI) | | | |
| 12 | in Pankreasnormalgewebe | Human protease E | trypsin | | |
| | überexprimiert | | | | |
| 14 | in Pankreasnormalgewebe | Homolog zu Trypsin precursor | | | |
| | überexprimiert | | | | |
| 15 | in Pankreasnormalgewebe | Humanes Chymotrypsinogen | TRYPSIN_{—} | 16q24.1-q24.2 | CHLC:GATA71F09 |
| | überexprimiert | | CATAL | | |
| 16 | in Pankreasnormalgewebe | Homo sapiens colipase (CLPS) | Colipase | 6p21.2-p21.31 | D6S439-D6S291 |
| | überexprimiert | | | | |
| 17 | in Pankreasnormalgewebe | caldecrin = serum calcium-decreasing factor | TRYPSIN_{—} | 1p36.13 | AFMa127zc9- |
| | überexprimiert | | CATAL | | AFMa232zb9 |
| 18 | in Pankreasnormalgewebe | Human pancreatic zymogen granule membrane | zona_pellucida | 16p11.2 | WI-3768 |
| | überexprimiert | protein GP-2 | | | |
| 19 | in Pankreasnormalgewebe | Humanes IAPP | 71m_2 | 12p12.3 | WI-7377-D12S1669 |
| | überexprimiert | | | | |
| 21 | in Pankreasnormalgewebe | H. sapiens mRNA for chymotrypsin-like | PRO_RICH | 16q22.3-q23.1 | WI-9392 |
| | überexprimiert | protease CTRL-1 | | | |
| 24 | in Pankreasnormalgewebe | Humanes Homolog zu D. melanogasler | EF_HAND_2 | | |
| | überexprimiert | Calbindin-32 | | | |
| 25 | in Pankreasnormalgewebe | Humanes Homolog zu R. norvegicus syncollin | | 19q13.2 | SHGC-8810-SHGC- |
| | überexprimiert | | | | 37129 |
| 27 | in Pankreasnormalgewebe | Mus musculus unknown protein precursor | CUB; zona_{—} | | |
| | überexprimiert | | pellucida | | |
| 28 | in Pankreasnormalgewebe | unbekannt | | | |
| | überexprimiert | | | | |
| 29 | in Pankreasnormalgewebe | unbekannt | | 16p12.3-p12.2 | D16S3045-SHGC- |
| | überexprimiert | | | | 6042 |
| 30 | in Pankreasnormalgewebe | unbekannt | | 16p12.3-p12.2 | D16S3045-SHGC- |
| | überexprimiert | | | | 6042 |
| 31 | in Pankreasnormalgewebe | unbekannt | trypsin | 16q23.1 | SHGC-14629- |
| | überexprimiert | | | | SHGC-8141 |
| 35 | in Pankreasnormalgewebe | unbekannt | PRO_RICH | 7q32.3-q33 | SHGC-30423- |
| | überexprimiert | | | | AFM183xe11 |
| 36 | in Pankreasnormalgewebe | unbekannt | PRO_RICH | 16p12.3-p12.2 | D16S3045-SHGC- |
| | überexprimiert | | | | 6042 |
| 37 | in Pankreasnormalgewebe | unbekannt | | 10q25.1-q25.3 | AFM249vf1-SHGC- |
| | überexprimiert | | | | 14812 |
| 67 | in Pankreasnormalgewebe | Verlängerung zu Seq ID: 27 | | | |
| | überexprimiert | | | | |

TABLE II

| | |
|---|---|
| DNA Seq. ID. No.: | Peptide Seq. ID. No.: |
| DNA Seq ID No: | Peptid Seq ID No: |

| | |
|---|---|
| 14 | 68 |
| | 69 |
| 24 | 39 |
| | 40 |
| 25 | 41 |
| | 42 |
| | 43 |
| 27 | 44 |
| 28 | 45 |
| 29 | 46 |
| 30 | 47 |
| | 48 |
| | 49 |
| 31 | 50 |
| | 51 |
| | 52 |
| 35 | 56 |
| | 57 |
| | 58 |
| 36 | 59 |
| | 60 |
| | 61 |
| 37 | 62 |
| | 63 |
| 67 | 70 |
| | 71 |

The invention nucleic acid sequences Seq. ID No. 1 to Seq. ID No. 37 and 67 of the determined candidate genes and the determined amino acid sequences Seq. ID No. 39 to Seq. ID Nos. 63 and 68-71 are described in the following sequence protocol.

For Information Only

Codes used for identifying PCT member countries on the head sheets of the publications of international applications according to the PCT.

AL Albania

AM Armenia

AT Austria

AU Australia

AZ Azerbaijan

BA Bosnia-Herzegovina

BB Barbados

BE Belgium

BF Burkina Faso

BG Bulgaria

BJ Benin

BR Brazil

BY Belarus

CA Canada

CF Central African Republic

CG Congo

CH Switzerland

CI Ivory Coast

CM Cameroon

CN China

CU Cuba

CZ The Czech Republic

DE Germany

DK Denmark

EE Estonia

ES Spain

FI Finland

FR France

GA Gabon

GB United Kingdom

GE Georgia

GH Ghana

GN Guinea

GR Greece

HU Hungary

IE Ireland

IL Israel

IS Iceland

IT Italy

JP Japan

KE Kenya

KG Kyrgyzstan

KP Democratic People's Republic of Korea

KR Republic of Korea

KZ Kazachstan

LC St. Lucia

LI Liechtenstein

LK Sri Lanka

LR Liberia

LS Lesotho

LT Lithuania

LU Luxembourg

LV Latvia

MC Monaco

MD Republic of Moldova

MG Madagascar

MK the former Yugoslavian Republic of Macedonia

ML Mali

MN Mongolia

MR Mauritania

MW Malawi

MX Mexico

NE Niger

NL The Netherlands

NO Norway

NZ New Zealand

PL Poland

PT Portugal

RO Romania

RU Russian Federation

SD Sudan

SE Sweden

SG Singapore

SI Slovenia

SK Slovakian Republic

SN Senegal

SZ Swaziland

TD Chad

TG Togo

TJ Tajikistan

TM Turkmenistan

TR Turkey

TT Trinidad and Tobago

UA The Ukraine

UG Uganda

US United States of America

UZ Uzbekistan

VN Vietnam

YU Yugoslavia

ZW Zimbabwe

1. A nucleic acid sequence that codes a gene product or a portion thereof, comprising
a) a nucleic acid sequence, selected from the group Seq. Id nos. 14, 24, 25, 27-31, 35-37 and 67,
b) an allelic variation of the nucleic acid sequences named under a)
or
c) a nucleic acid sequence that is complementary to the nucleic acid sequences named under a) or b):

2. A nucleic acid sequence according to one of the sequences Seq. ID Nos. 14, 24, 25, 27-31, 35-37, 67 or a complementary or allelic variant thereof.

3. Nucleic acid sequence Seq. ID Nos. 2-37 and 67, characterized in that it is expressed elevated in normal pancreatic tissue.

4. BAC, PAC and cosmid clones containing functional genes and their chromosomal localization according to sequences Seq. ID Nos. 2-37 and 67 for use as vehicles for gene transfer.

5. A nucleic acid sequence according to claims 1 to 4, wherein it has 90% homology to a human nucleic acid sequence.

6. A nucleic acid sequence according to claims 1 to 4, wherein it has 95% homology to a human nucleic acid sequence.

7. A nucleic acid sequence comprising a portion of the nucleic acid sequences named in claims 1 to 6, in such a sufficient amount that they hybridize with the sequences according to claims 1 to 6.

8. A nucleic acid sequence according to claims 1 to 7, wherein the size of the fragment has a length of at least 50 to 4500 bp.

9. A nucleic acid sequence according to claims 1 to 7, wherein the size of the fragment has a length of at least 50 to 4000 bp.

10. A nucleic acid sequence according to one of claims 1 to 9, which codes at least one partial sequence of a bioactive polypeptide.

11. An expression cassette, comprising a nucleic acid fragment or a sequence according to one of claims 1 to 9, together with at least one control or regulatory sequence.

12. An expression cassette, comprising a nucleic acid fragment or a sequence according to claim 11, in which the control or regulatory sequence is a suitable promoter.

13. An expression cassette according to one of claims 11 and 12, wherein the DNA sequences located on the cassette code a fusion protein, which comprises a known protein and a bioactive polypeptide fragment.

14. Use of nucleic acid sequences according to claims 1 to 10 for producing full-length genes.

15. A DNA fragment, comprising a gene, that can be obtained from the use according to claim 14.

16. Host cell, containing as the heterologous part of its expressible genetic information a nucleic acid fragment according to one of claims 1 to 10.

17. Host cell according to claim 16, wherein it is a prokaryotic or eukaryotic cell system.

18. Host cell according to one of claims 16 or 17, wherein the prokaryotic cell system is E. coli and the eukaryotic cell system is an animal, human or yeast cell system.

19. A process for producing a polypeptide or a fragment, wherein the host cells according to claims 16 to 18 are cultivated.

20. An antibody that is directed against a polypeptide or a fragment that is coded by the nucleic acids of sequences Seq. ID Nos. 2-37 and 67, which can be obtained according to claim 19.

21. An antibody according to claim 20, wherein it is monoclonal.

22. An antibody according to claim 20, wherein it is a phage display antibody.

23. Polypeptide partial sequences according to sequences Seq. ID Nos. 39-63 and 68-71.

24. Polypeptide partial sequences according to claim 23, with at least 80% homology to these sequences.

25. A polypeptide that is known from a phage display and that can bind to the polypeptide-partial sequences according to claim 23.

26. Polypeptide partial sequences according to claim 23, with at least 90% homology to these sequences.

27. Use of polypeptide partial sequences according to sequences Seq. ID Nos. 39-63 and 68-71 as tools for finding active ingredients against pancreatic tumors.

28. Use of nucleic acid sequences according to sequences Seq. ID Nos. 2-37 and 67 for expression of polypeptides that can be used as tools for finding active ingredients against pancreatic tumors.

29. Use of nucleic acid sequences Seq. ID Nos. 2-37 and 67 in sense or antisense form.

30. Use of polypeptide partial sequences Seq. ID Nos. 39-63 and 68-71 as pharmaceutical agents in gene therapy for treatment of pancreatic tumors.

31. Use of polypeptide partial sequences Seq. ID Nos. 39-63 and 68-71 for the production of a pharmaceutical agent for treatment of pancreatic tumors.

32. Pharmaceutical agent, containing at least one polypeptide partial sequence Seq. ID Nos. 39-63 and 68-71.

33. A nucleic acid sequence according to claims 1 to 10, wherein it is a genomic sequence.

34. A nucleic acid sequence according to claims 1 to 10, wherein it is an mRNA sequence.

35. Genomic genes, their promoters, enhancers, silencers, exon structure, intron structure and their splice variants, that can be obtained from cDNAs of sequences Seq. ID Nos. 2-37 and 67.

36. Use of the genomic genes according to claim 33, together with suitable regulatory elements.

37. Use according to claim 36, wherein the regulatory element is a suitable promoter and/or enhancer.

38. A nucleic acid sequence according to claims 1 to 7, wherein the size of the fragment has a length of at least 300 to 3500 bp.
